(19) United States
(12) Patent Application Publication
(10) Pub. No.: US 2009/0042195 A1
(43) Pub. Date: Feb. 12, 2009

(54) METHODS AND SYSTEMS FOR SCREENING FOR AND DIAGNOSING DNA METHYLATION ASSOCIATED ABNORMALITIES AND SEX CHROMOSOME ANEUPLOIDIES

(76) Inventors: Bradford Coffee, Decatur, GA (US); Kasinathan Muralidharan, Chantilly, VA (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/089,327

(22) PCT Filed: Oct. 10, 2006
(86) PCT No.: PCT/US06/39668

§ 371 Date: Sep. 4, 2008

Related U.S. Application Data
(60) Provisional application No. 60/724,633, filed on Oct. 7, 2005.

(51) Int. Cl. C12Q 1/68

(52) U.S. Cl. 435/6

(57) ABSTRACT

Methods and systems for population screening and diagnostics are provided. In particular methods and systems for population screening of individuals for genetic disorders due to alterations in DNA methylation and for the diagnostic testing for such disorders are provided.

### CROSS-REFERENCE TO RELATED APPLICATION

This application claims priority to copending U.S. provisional patent application Ser. No. 60/724,633, entitled “Methods and Systems for Screening for DNA Methylation Associated Abnormalities and Sex Chromosome Aneuploidies” filed on Oct. 7, 2005, which is entirely incorporated herein by reference.

### STATEMENT REGARDING FEDERALLY SPONSORED RESEARCH OR DEVELOPMENT

This invention was made with government support under grant number MM-0937-06/06 awarded by the Centers for Disease Control and Prevention. The government has certain rights in the invention.

### FIELD OF THE INVENTION(S)

The present disclosure relates to the field of population screening and diagnostics, particularly to the population screening of individuals for genetic disorders due to alterations in DNA methylation and diagnostic testing for such disorders. More particularly, it relates to screening for disorders such as, but not limited to, fragile X syndrome, sex chromosome aneuploidies, Prader-Willi syndrome, Angelman syndrome, autism due to maternally inherited 15q11-q13 duplications, Beckwith-Weidemann syndrome and Silver-Russell syndrome, which are characterized by aberrant patterns of DNA methylation, and to diagnostic testing for such disorders.

### BACKGROUND

The precise regulation of gene expression, controlled in large part by epigenetic modifications to chromatin, is essential for normal development and function in many organisms, including humans. DNA methylation and histone modifications are essential components in the establishment of the transcriptional state of many eukaryotic genes. The best understood of these epigenetic modifications is DNA methylation, which occurs primarily at cytosines located 5′ to guanosine in the CpG dinucleotide. This modification, when found in CpG rich areas, known as CpG islands, located in the promoter regions of many genes, is associated with transcriptional repression. While almost all gene-associated islands are protected from methylation on autosomal chromosomes, extensive methylation of CpG islands has been associated with transcriptional silencing of imprinted genes (genes that are differentially expressed based on their parent of origin). DNA methylation also plays an essential role in the maintenance of the transcriptionally silent, inactive X chromosome in females.

Alterations in the normal patterns of DNA methylation are associated with diseases such as fragile X syndrome, Prader-Willi, Angelman, Silver-Russell syndrome and Beckwith-Wiedemann syndromes. In addition, abnormal DNA methylation can also be associated with disorders of sex chromosomes, such as Turner, Klinefelter syndrome, other sex chromosome aneuploidies and their variants.

Southern blot analysis using methylation sensitive restriction enzymes is a well-established method for detecting methylation of DNA sequences and differentiating between methylated and unmethylated homologues. The main disadvantages of this technique are the time taken to perform the procedure, the large amounts of DNA necessary for analysis, and the use of radioisotopes.

Alternative methods to test for alterations in DNA methylation are based on conventional methylation-sensitive PCR using sodium bisulfite treated DNA as a template. However, these methods employ purified DNA samples, which requires additional time and is therefore not amenable to population screening for methylation disorders. Additionally, methods employing conventional methylation-specific PCR require two sets of PCR primers, one set specific for the methylated strand and a second set specific for the unmethylated strand. This approach often results in unequal PCR amplification of the methylated and unmethylated templates and is not suited for differentiating levels of methylation.

### SUMMARY

The present disclosure provides methods and assays for detecting and quantifying methylation of nucleic acid-containing samples. The present disclosure further provides methods and assays for screening both male and female members of a population for disorders associated with abnormal DNA methylation. Methods and assays of the present disclosure are able to detect the presence of such disorders in a subject directly from a crude DNA extract from blood or tissue sample from the subject, without isolation and/or purification of the DNA from the sample.

Embodiments of methods of detecting and quantifying abnormal methylation in a nucleic acid-containing sample include contacting a nucleic acid containing sample from a subject with an agent that modifies unmethylated cytosine; amplifying a target nucleic acid sequence; and quantifying an amount of a methylated version of the target nucleic acid sequence and an amount of an unmethylated version of the target nucleic acid sequence with probes capable of distinguishing between the target nucleic acid sequence containing methylated cytosines and the target nucleic acid sequence not containing methylated cytosines. In embodiments of the methods of the present disclosure the cytosines are converted to uracil and the agent is sodium bisulfate, optionally in combination with hydroquinone. In preferred embodiments, real-time PCR is used for the amplification and quantification of the target nucleic acid. In embodiments of the present disclosure the probes include a first probe specific for the unmethylated target nucleic acid sequence and having a first reporter molecule and a quencher molecule and a second probe specific for the methylated target nucleic acid sequence and having a second reporter molecule and a quencher molecule, where the first and second reporter molecules are distinguishable.

Embodiments of the present disclosure include screening for a condition associated with abnormal methylation of a target nucleic acid sequence in a specific gene indicated by the amount of a methylated version of the target nucleic acid. In embodiments, the condition is selected from at least one of the following: Fragile X syndrome, Prader-Willi syndrome, Angelman syndrome, autism, Silver Russell syndrome, Beckwith-Wiedemann syndrome, and disorders resulting from sex chromosome aneuploidies, such as Klinefelter syndrome and its variants, Turner syndrome and its variants, XXX syndrome and its variants, and XYY syndrome and its variants. In embodiments of the present disclosure methods and assays for screening for and/or diagnosing fragile X syndrome in males, mosaic males, and females are provided.

Embodiments of the methods of the present disclosure also provide for screening samples from more than one individual (e.g., more than 10, more than 50, and more than 100) for abnormal methylation of a target nucleic acid sequence in a single assay. In embodiments, the methylation status of more than one target nucleic acid sequence can be tested in a single assay. In some embodiments of the disclosure, methods and assays are provided for analyzing and quantifying DNA methylation and CGG tract repeats in a single assay.

Embodiments of the present disclosure provide methods of screening members of a population for conditions associated with sex chromosome abnormalities including: obtaining samples from one or more subjects, regardless of whether said subjects present any symptoms of conditions associated with sex chromosome abnormalities; contacting the samples with an agent that modifies unmethylated cytosine; quantifying an amount of a methylated version of at least one target nucleic acid sequence and an amount of an unmethylated version of the at least one target nucleic acid sequence with probes capable of distinguishing between the target nucleic acid sequence containing methylated cytosines and the target nucleic acid sequence not containing methylated cytosines; and quantifying the number of X and Y chromosomes present in the sample.

The present disclosure also includes kits for screening subjects for conditions associated with abnormal DNA methylation. Embodiments of kits according to the present disclosure include a primer pair specific for a first nucleic acid sequence in the promoter region of a gene associated with the condition, a probe pair specific for a second nucleic acid sequence in the promoter region of the gene, wherein the first and second nucleic acid sequences are different, and wherein the probe comprises a first probe specific for an unmethylated version of the second nucleic acid sequence and a second probe specific for a methylated version of the second nucleic acid sequence. In an embodiment of a kit of the present disclosure for screening for fragile X syndrome and sex chromosome aneuploidies, the kit includes a primer pair specific for a first nucleic acid sequence in the promoter region of the FMR1 gene, a probe pair specific for a second nucleic acid sequence in the promoter region of the FMR1 gene, wherein the first and second nucleic acid sequences are different, and wherein the probe comprises a first probe specific for an unmethylated version of the second nucleic acid sequence and a second probe specific for a methylated version of the second nucleic acid sequence. The kit optionally also includes a primer pair specific for a first nucleic acid sequence of the SRY gene and a probe pair specific for a second nucleic acid sequence of the SRY gene, where the first and second nucleic acid sequences are different.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### BRIEF DESCRIPTION OF THE DRAWINGS

The disclosure can be better understood with reference to the following drawings. The components in the drawings are not necessarily to scale, emphasis instead being placed upon clearly illustrating the principles of the present disclosure.

FIG. 1A illustrates the general design and location of amplification primers and Taqman probes to target a DNA sequence for methylation analysis. As shown, the probe for the normal (or unmethylated) DNA is labeled with FAM, and the probe for the methylated DNA is labeled with HEX to allow differentiation and quantification of the two in real-time PCR. This is illustrated in more detail in FIG. 1B for the promoter region of the FMR1 gene (SEQ ID NO: 17). The location of the forward and reverse primers is indicated by underline, the location beginning of the coding region of the FMR1 gene is indicated with an arrow, and the location of the probe sequences is enclosed by brackets.

FIG. 2 illustrates amplification plots for methylated (FAM labeled Taqman probe) and unmethylated (HEX labeled Taqman probe) FMR1 DNA from a sample from a normal female.

FIG. 3 is a bar graph illustrating the amounts of methylated FMR1 DNA samples from screening nine male subjects (1 normal male, 2 positive fragile X males, 3 males mosaic for fragile X, and 3 Klinefelter males) according to methods of the present disclosure. The results indicate that quantitative methylation sensitive PCR methods of the present disclosure can detect FMR1 methylation that is due to the presence of CGG repeat tract expansion (fragile X) or the presence of more than one X chromosome (Klinefelter syndrome and its variants).

FIG. 4 illustrates amplification plots for samples from 88 male subjects (obtained from dried blood spots), two of which were mosaic for fragile X and 86 of which were normal (8 wells were reserved for control samples). The right-hand graph illustrates the amplification plot of unmethylated FMR1 DNA, while the left-hand graph illustrates the amplification plot of methylated FMR1 DNA, showing the clear identification of the mosaic fragile X subjects.

FIG. 5 illustrates amplification plots of FMR1 DNA from 96 subjects showing the clear detection of a single mosaic fragile X male sample from among 95 normal male samples.

FIG. 6 is a bar graph illustrating the methylation indices for FMR1 DNA from samples from 33 females harboring full fragile X mutations and from 13 negative controls.

FIG. 7 is a bar graph illustrating methylation indices (MI) for SNRPN DNA from samples from a normal subject, a subject with Angelman syndrome (due to deletion of maternal 15q11-13), a subject with a maternally inherited duplication of 15q11-13 (a form of autism), a subject with a maternally inherited triplication of 15q11-13 (a form of autism), and a subject with Prader-Willi Syndrome (due to deletion of the paternal copy of 15q11-13).

FIG. 8 illustrates the imprinted genes located at the maternal and paternal copies of 11p15. CTCF is the chromatin insulator protein CCTC-binding factor. KCNQ10T1 (or Lit1) is located on the antisense strand in intron 10 of the KCNQ1 gene. CH₃ indicates sites of DNA methylation on the different parental alleles. The differentially methylated regions (DMR1 and DMR2) are illustrated along with other genes within these regions.

FIG. 9 illustrates amplification plots and standard curves for both methylated and unmethylated probes targeting the KCNQ10T1 promoter. The amount of sodium bisulfite-treated DNA for each curve is indicated, as well as the crossing threshold for the methylated DNA amplification chart and the unmethylated DNA amplification chart. The standard curves are illustrated to the right of each amplification chart (known standards=circles; unknown samples=X's).

FIG. 10A illustrates methylation indices for 15 normal individuals tested via quantitative methylation sensitive PCR (using Taqman MSP) (light gray bars) and tested via Southern analysis (dark gray bars). Samples 9 to 15 were tested only by Q-MSP. FIG. 10B illustrates the average MI of 92 negative samples having a normal range of 0.40 to 0.64 (dark gray box) (average MI: 0.52; standard deviation: 0.06). FIG. 10B also compares southern analysis (dark gray bars) and Q-MSP (light gray bars) analysis of the methylation indices for 14 patients diagnosed with Beckwith-Wiedemann syndrome (BWS).

FIG. 11A is a bar graph illustrating methylation indices of CTCF6 DNA for 11 healthy individuals by Q-MSP (Taqman), showing a mean MI of 0.52 with a SD of 0.05, resulting in a normal range of 0.42 to 0.62. Assessment of 91 negative samples resulted in an average MI of 0.49 with a SD of 0.08, resulting in a normal range of 0.33 to 0.65, shown as the dark gray horizontal box on FIG. 11B. FIG. 11B illustrates a comparison of the MI's obtained by southern analysis (dark gray bars) and Q-MSP (light gray bars) for the H19 promoter in 27 subjects clinically diagnosed with BWS.

FIG. 12 illustrates simultaneous analysis of methylation of DMR1 (dark gray bars) and DMR2 (light gray bars) in a healthy individual and in an infant with a duplication of 11p15.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere. Experimental hypoxia was obtained by growing cells in culture medium in an incubator under an environment of 1% partial pressure of oxygen unless otherwise indicated.

Embodiments of the present disclosure will employ, unless otherwise indicated, techniques of synthetic organic chemistry, biochemistry, molecular biology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

### DEFINITIONS

The term “nucleic acid” or “polynucleotide” is a term that generally refers to a string of at least two base-sugar-phosphate combinations. As used herein, the term includes deoxyribonucleic acid (DNA) and ribonucleic acid (RNA) and generally refers to any polyribonucleotide or polydeoxyribonucleotide, which may be unmodified RNA or DNA or modified RNA or DNA. RNA may be in the form of an tRNA (transfer RNA), snRNA (small nuclear RNA), rRNA (ribosomal RNA), mRNA (messenger RNA), anti-sense RNA, RNAi, siRNA, and ribozymes. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid sequence” or “oligonucleotide” also encompasses a nucleic acid or polynucleotide as defined above.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein.

The term also includes PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotides” as that term is intended herein.

As used herein “modifies” refers to the conversion of an unmethylated cytosine to another nucleotide, which distinguishes the unmethylated from the methylated cytosine. Preferably, the agent modifies unmethylated cytosine to uracil. Preferably, the agent used for modifying unmethylated cytosine is sodium bisulfite, however, other agents that similarly modify unmethylated cytosine, but not methylated cytosine, can also be used in the method of the disclosure. Sodium bisulfite (NaHSO₃) reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by Taq polymerase and therefore upon PCR, the resultant product contains cytosine only at the position where 5-methylcytosine occurs in the starting template DNA.

As used herein “primer” generally refers to polynucleotides (e.g., oligonucleotides) of sufficient length and appropriate sequence so as to provide specific initiation of polymerization on a significant number of nucleic acids in the polymorphic locus. Specifically, the term “primer” refers to a polynucleotide sequence including two or more deoxyribonucleotides or ribonucleotides, preferably more than three, and most preferably more than 8, which sequence is capable of initiating synthesis of a primer extension product, which is substantially complementary to a polymorphic locus strand. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded. If double stranded, the primer is first treated to separate its strands before being used to prepare extension products. Preferably, the primer is an oligo deoxyribonucleotide. The primer is sufficiently long to prime the synthesis of extension products in the presence of the inducing agent for polymerization. The exact length of primer depends on many factors, including temperature, buffer, and nucleotide composition. The oligonucleotide primer typically contains 12-20 or more nucleotides, although it may contain fewer nucleotides.

Primers of the present disclosure are designed to be “substantially” complementary to each strand of the genomic locus to be amplified and include the appropriate G or C nucleotides as discussed above. Thus, the primers are sufficiently complementary to hybridize with their respective strands under conditions that allow the agent for polymerization to perform. In other words, the primers should have sufficient complementarity with the 5′ and 3′ flanking sequences to hybridize therewith and permit amplification of the genomic locus.

Oligonucleotide primers of the present disclosure are employed in the amplification process, which is an enzymatic chain reaction that produces exponential quantities of target locus relative to the number of reaction steps involved. Typically, one primer is complementary to the negative (−) strand of the locus and the other is complementary to the positive (+) strand. Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (e.g., the target locus sequence) defined by the primer. The product of the chain reaction is a discrete nucleic acid duplex with termini corresponding to the ends of the specific primers employed.

The oligonucleotide primers of the present disclosure may be prepared using any suitable method, such as conventional phosphotriester and phosphodiester methods or automated embodiments thereof. In one such automated embodiment, diethylphosphoramidites are used as starting materials and may be synthesized as described by Beaucage, et al. (Tetrahedron Letters, 22:1859-1862, 1981, which is hereby incorporated by reference in its entirety). One method for synthesizing oligonucleotides on a modified solid support is described in U.S. Pat. No. 4,458,066, which is hereby incorporated by reference herein.

As used herein “probes” generally refer to polynucleotides (e.g., oligonucleotides) of sufficient length and appropriate sequence designed for binding to a target DNA or RNA for a variety of purposes (e.g., identification of a specific target sequence). As used herein “probes” differ from primers in that although probes may sometimes be capable of priming if used in an amplification process, the probes of the present disclosure are not used for priming purposes in an amplification process according to the methods of the present disclosure, but are instead used for identifying/distinguishing specific target sequences (e.g., in a real-time PCR process). In embodiments of the present disclosure the probes are Taqman probes for use in real time PCR. An embodiment of a probe set for use according to the present disclosure for distinguishing between a methylated and unmethylated target nucleic acid sequence includes a first and second probe, where the first probe is specific for the unmethylated target sequence and has a first reporter molecule (e.g., a reporter dye such as a fluorophore), and the second probe is specific for the methylated target sequence and has a second reporter molecule that is distinguishable from the first reporter molecule. The probes also usually include a quencher (e.g., a quencher dye) for suppressing the detectable signal of the reporter molecule in the absence of the target sequence. In this way, detection of the signal of the reporter molecule indicates the presence of the target sequence in the sample.

Also, although “primers” as used herein, may be sometimes be labeled for detection purposes in addition to amplification purposes, when used as such they are referred to herein as “labeled primers”.

“Real-time PCR” and “kinetic PCR” are used herein to refer to a polymerase chain reaction (PCR) technique in which probes, as described above, are included in the reaction mixture during the amplification process, allowing real-time detection and quantification of target products of the amplification process. The detection and quantification of the amplified target sequence(s) is achieved by the use of real-time PCR instrumentation capable of detecting and quantifying the signal from the probes. One example of real-time PCR is the Taqman technique, which is known to those of skill in the art, and described in greater detail in the examples below.

As used herein “population screening” and “screening” are methods used to identify, within a population or group of individuals, asymptomatic or presymptomatic individuals at risk of developing a disorder, whereas “diagnosis” generally refers to the process of testing symptomatic individuals for the presence of a disorder. In contrast to clinical diagnostic testing, where typically only symptomatic individuals are tested, in population screening all individuals within a population or other defined group are screened for a disorder. If an individual screens positive, a follow-up visit is scheduled where additional samples are obtained for confirmatory testing. Thus, the primary goal of screening is not the clinical diagnosis of disease, but to identify those who are at risk. After confirmation appropriate medical management decisions can then be instituted to prevent or ameliorate symptoms of the disease. Newborn screening for genetic disorders is such a program that identifies individuals at risk of metabolic genetic disorders. This disclosure relates to the population screening of individuals for alterations in normal patterns of DNA methylation.

### DESCRIPTION

The present disclosure provides methods and assays that detect changes in DNA methylation that lead to disease. The methods and assays of the present disclosure detect such changes in DNA methylation directly from a tissue or blood sample in a high-throughput format and without the need for isolation of the DNA. Methods and assays of the present disclosure also allow quantitative analysis of the DNA methylation status of a nucleic acid-containing sample, which provides more detailed diagnostic information as well as the ability to diagnose and screen for disorders not identifiable by mere qualitative detection of DNA methylation. This disclosure describes methods used to test for alterations in DNA methylation for such disorders within the population screening paradigm. The present disclosure also describes novel methods and kits for testing for DNA methylation associated disorders that combine DNA methylation analysis and CGG repeat tract analysis in a single assay. Several disorders characterized by abnormal DNA methylation are described below.

Diseases Associated with Altered Patterns of DNA Methylation

Fragile X Syndrome

Fragile X syndrome is the most common inherited form of mental retardation and developmental disability. The prevalence of fragile X syndrome in males is estimated to be somewhere between 1 in 3700 to 1 in 8900. The prevalence among females is expected to be half that found in males. Males affected with fragile X syndrome suffer from moderate mental retardation and often exhibit characteristic physical features and behavior. In addition, autistic-like behaviors are commonly seen in males with fragile X syndrome. Generally speaking, the problems experienced by fragile X females, who typically suffer from milder mental retardation, are less severe than their male counterparts.

The primary cause of fragile X syndrome is the expansion of a CGG trinucleotide repeat in the promoter region of the FMR1 gene. This expansion leads to the silencing of FMR1 transcription and absence of the FMR1 gene product, FMRP. In the general population the average number of units in this tract is 30 CGG repeats, with a range from 0 to 50 CGGs. Repeat tracts containing between 51 and 200 repeats are referred to as pre-mutations. These individuals are not affected with fragile X syndrome but may suffer from a distinct adult onset disorder termed fragile X-associated tremor/ataxia syndrome (FXTAS). In addition, in females there is an increased incidence of premature ovarian failure in carriers of pre-mutation sized alleles. When FMR1 alleles in pre-mutation range are transmitted maternally to offspring there is high likelihood for expansion of the CGG repeat tract to greater than 200 CGGs. Expansions of >200 CGG repeats, such as up to several thousand repeats, trigger aberrant methylation of the FMR1 DNA and associated changes in chromatin structure that leads to its transcriptional silencing. Therefore, most affected individuals do not express the FMR1 mRNA. There are individuals who have CGG repeat tract expansions that are above and below the 200 repeat threshold who have incomplete methylation of the FMR1 gene. These individuals are referred to as “mosaics” and are typically less affected than full expansion patients.

Molecular diagnosis of this disorder is based on CGG repeat number determination and methylation analysis of the FMR1 gene. PCR analysis has been used to determine the number of repeats, and Southern analysis has been used to determine the methylation status. A methylation-specific PCR process to diagnose males with fragile X syndrome having full mutations has been described (U.S. Pat. No. 6,143,504, which is incorporated by reference herein). However, a method for detecting females with fragile X syndrome and/or males mosaic for the disorder has not been available. A PCR based method based on amplifying CGG repeats has been described for identifying Fragile-X, but, since this method depends on the non-amplification of a full mutation as an indicator for fragile X, it is error prone.

### Sex Chromosome Abnormalities

Sex chromosome abnormalities are the most common chromosome abnormalities in newborns, with an overall incidence estimated to be 1 in 500 (Nielsen et al. 1991). These disorders, Turner syndrome (45,X), Klinefelter syndrome (47, XXY), Trisomy X (47,XXX) and other variants, are characterized by the presence of an abnormal number of X chromosomes. Although the phenotype in these disorders is relatively mild compared to other genetic disorders, there is substantial medical benefit in identifying individuals with sex chromosome abnormalities during the newborn period. Individuals with Turner syndrome are at risk for life-threatening cardiac defects. In addition, mosaic Turner patients who carry portions of the Y chromosome are at risk for developing gonadoblastoma. The phenotype in Klinefelter syndrome is less severe, but patients often display learning difficulties and would benefit from early educational intervention.

The primary method for detecting these disorders is karyotype analysis performed in cytogenetic laboratories. However, this method is not practical for high-throughput population screening. FMR1 DNA methylation analysis can distinguish between the active and inactive X chromosomes in normal females; therefore, DNA methylation analysis can be used in the methods and systems of the present disclosure to detect sex chromosome abnormalities. For example, Klinefelter syndrome (47,XXY) will be detected as a male with two FMR1 genes, one active (unmethylated) and one inactive (methylated), a normal female pattern. Females with a single unmethylated X chromosome, Turner Syndrome, will have a normal male pattern. In individuals with more than 2 X chromosomes (47, XXX, 48XXXY, etc.) inactivation of all but one of the X chromosomes occurs to compensate for dosage. Calculation of the methylated:unmethylated FMR1 ratio would allow the determination of the number of X chromosomes in a sample and would lead to the identification of individuals with sex chromosome abnormalities.

Prader-Willi (PWS), Angelman (AS) Syndromes, and Autism Due to Maternally Inherited 15q11-13 Duplications

Both Prader-Willi and Angelman syndrome are due to defects in an imprinted region located at 15q11-q13. Prader-Willi syndrome (PWS) is characterized by severe hypotonia and feeding difficulties in early infancy. After infancy there is development of excessive eating behavior leading to morbid obesity, unless externally controlled. There is cognitive impairment present in all patients. Angelman syndrome (AS) is characterized by severe developmental delay, severe speech impairment, gait ataxia, and a unique behavior with an inappropriate happy demeanor that includes frequent laughing, smiling, and excitability.

PWS is caused by absence of the paternally derived Prader-Willi syndrome/Angelman syndrome (PWS/AS) region of chromosome 15 by one of several genetic mechanisms. Over 99% of patients with PWS have a diagnostic abnormality in the parent-specific methylation imprint within the Prader-Willi critical region. AS is caused by loss of the maternally imprinted UBE3A gene located in the 15q11.2-q13 region. There are five distinct mechanisms that lead to these disorders: 1) large cytogenetically visible deletions that are 3-5 mega bases in size, 2) chromosome 15 uniparental disomy (UPD) with maternal UPD resulting in PWS and paternal UPD causing AS, 3) imprinting defects-associated with loss of DNA methylation at the PW/AS imprinting center, 4) point mutation in the UBE3A gene causing AS and 5) other unknown mechanisms. Deletions and maternal UPD account for >99% PWS and can be detected by DNA methylation analysis. Greater than 70% of the cases of AS are due to defects that can be detected by DNA methylation analysis.

One of the most common causes of autism is maternally inherited duplication of 15q11-13. These duplications lead to overexpression of genes located in this segment of chromosome 15 leading to the autistic phenotype. It is estimated that 1-3% of cases of autism are due to this mechanism. Duplications of 15q11-13 can be interstitial or involve generation of extrachromosomal material (small marker chromosomes). Quantification of DNA methylation at SNRPN will not only identify individuals with Prader-Willi and Angelman Syndromes, but will identify individuals with duplications and triplications of 15q11-13 and will be able to determine the parent of origin of these copy number changes.

### Beckwith-Wiedemann (BWS) and Silver Russell (SRS) Syndromes

BWS is an overgrowth syndrome that is characterized by congenital malformations and tumor predisposition. BWS is due to disruption of imprinted gene expression at 11p15. The most common cause of BWS is loss of methylation at the Lit1 promoter, accounting for 50% of the cases. Paternal UPD accounts for 10-20% of cases. DNA methylation analysis of the Lit1 promoter will detect these defects. Loss of the adjacent imprinted IGF2 gene are found in 25-50% of cases with a small fraction of those associated with loss of H19 DNA methylation. In addition to BWS, isolated hemihyperplasia and Russell-Silver Syndrome have been associated with defects in DNA methylation and imprinted gene expression at 11p15.

### Methods, Assays and Kits for Detecting and Quantifying DNA Methylation

Briefly described, methods for screening members of a population for disorders associated with abnormal DNA methylation according to the present disclosure include the following general steps: obtaining a nucleic-acid containing sample from one or more subjects; contacting the sample with an agent that modifies unmethylated cytosines;

amplifying a target nucleic acid in the sample by PCR; and discriminating between and quantifying the methylated and unmethylated target nucleic acid. In embodiments of the present disclosure, discriminating between methylated and unmethylated DNA is accomplished by the use of a first and second set of primers, a first set specific for the unmethylated DNA and the second set specific for the methylated DNA. In a preferred embodiment, discriminating between methylated and unmethylated DNA and quantification of methylated and unmethylated DNA is accomplished by contacting the sample during the amplification process with at least a first and a second probe, where the first probe is specific for unmethylated DNA and the second probe is specific for methylated DNA and where the two probes are distinguishable (e.g., the label of the first probe produces a distinguishable signal from the signal produced by the second probe) and allow quantification of the relative amounts of methylated and unmethylated DNA.

In preferred embodiments of this method, the modification of unmethylated cytosines is the sodium bisulfite/hydroquinone mediated chemical conversion of cytosines in DNA to uracil. 5-methylcytosines in DNA are resistant to this conversion, thus allowing the distinction between methylated and unmethylated DNAs. The converted DNA is analyzed after treatment by methods such as, but not limited to methylation specific PCR primers, and, preferably, methylation specific probes for use in real-time PCR. If conventional methylation sensitive PCR is used, other detection methods may be used during or after the completion of the PCR reaction to determine the relative amounts of methylated and unmethylated DNAs in the original sample (e.g., capillary electrophoresis, or other separation techniques).

One of many advantages of the methods of the present disclosure is that these methods allow the use of crude of extracts directly from tissue samples, such as blood or buccal brush samples, without need for DNA isolation, as in other methods. This saves the time and expense of isolating and purifying the sample prior to treatment and analysis. In an embodiment of the present disclosure, the sample includes blood spots, similar to the ones used in newborn screening programs. An advantage to using dried blood spots is that the procedures for obtaining, cataloging, and storing dried blood spot samples are already in place at newborn screening laboratories.

In such an embodiment, the blood spot is punched out from the filter card on which the sample was obtained; a portion of the filter paper is boiled in water briefly to lyse the cells, releasing the DNA. This extract is used directly for the sodium bisulfite/hydroquinone treatment. This assay could also be applied to other tissues, such as buccal brush samples, to test for alterations in DNA methylation. The sample may also be treated with NaOH to denature the DNA after boiling but prior to the sodium bisulfite/hydroquinone treatment.

Another advantage of the methods and assays of the present disclosure is that any locus that has alterations in DNA methylation can be assessed by this method. In addition, multiple loci can be examined simultaneously for DNA methylation alterations. For example in males fragile X, Klinefelter, Prader-Willi, Angelman, maternally inherited 15q11-13 duplications associated with autism, Beckwith-Wiedemann and Silver-Russell syndromes could be assessed in one reaction. In females this assay could also be used to test for fragile X, Prader-Willi, Angelman and Beckwith-Wiedemann syndromes as well as X chromosome aneuploidies. Moreover, both qualitative and quantitative DNA methylation analysis can be obtained in the same assay.

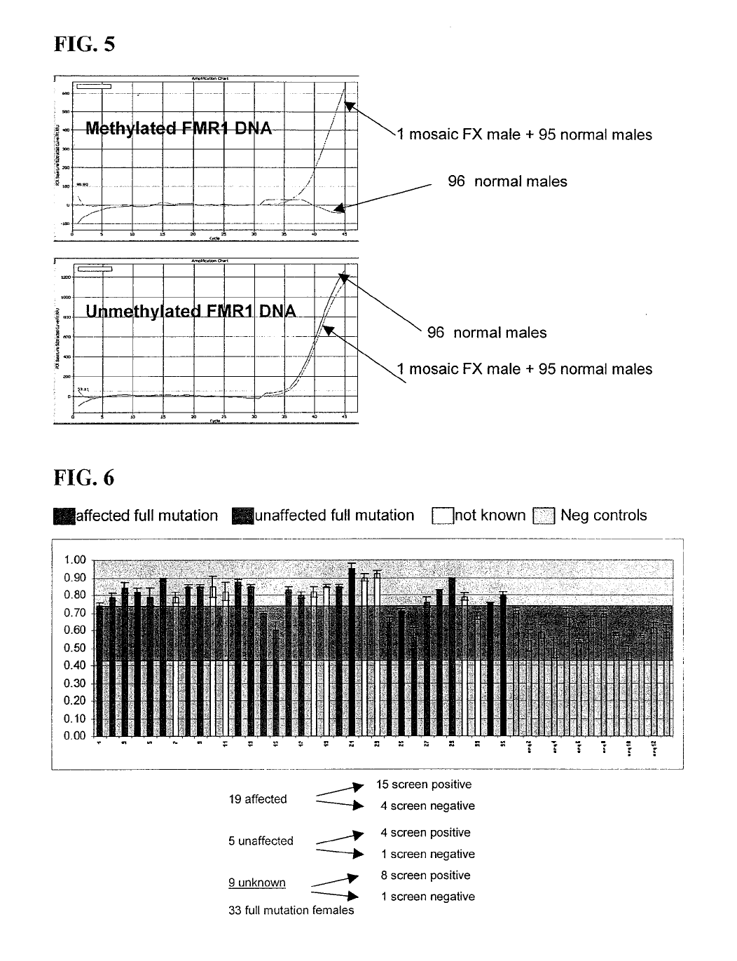

Additionally, methods according to the present disclosure can be used for high-throughput analysis. The extract preparation, sodium bisulfite/hydroquinone treatment, clean-up and quantitative methylation sensitive PCR analysis using fluorescently labeled primers can be done in 96 or 384 well formats allowing for the processing of large numbers of samples. For instance, for fragile X syndrome and Klinefelter syndrome screening in males, pools of dried blood samples (up to 100 at a time) can be assayed simultaneously for the presence of methylated FMR1 DNA (as described in example 1 below and illustrated in FIG. 5). If no methylated FMR1 DNA is detected in the pooled set, then all 100 samples are screened negative for fragile X and Klinefelter syndromes. However, if methylated FMR1 DNA is detected then the pooled samples can be analyzed individually for these disorders. Since the incidence of these disorders is 1 in 4000 for fragile X and 1 in 1000 for Klinefelter syndrome, the pooling of male samples allows for rapid screening of a large set of samples at a significantly reduced cost.

In one embodiment of the present disclosure, a PCR-based method for the analysis of methylation of the FMR1 gene is provided to determine if a male or female subject has fragile X syndrome. This method may be performed diagnostically, after presence of the disease is suspected, or may be used as a screening tool, to screen members of a population for presence of the disorder, before symptoms of the disorder have been manifested. For instance, the method may be used for systematic newborn screening, as is known for other disorders.

The methods of the present disclosure provide the ability to detect fragile X females in the population and mosaic males by quantitating the ratio of methylated and unmethylated FMR1 alleles, as described in greater detail in the examples below. This quantitation can be done with either a kinetic method or by end point analysis. Examples of kinetic methods are real-time PCR, pyro sequencing, etc (e.g., by the use of quantitative methylation specific PCR employing methylation-specific probes). An example of end-point analysis is separation and quantitation of fluorescently labeled MSP products (e.g., conventional methylation specific PCR using labeled primers and followed by capillary electrophoresis for quantitative end-point analysis).

In embodiments of the present disclosure, methylation-specific PCR is used to analyze the sodium bisulfite/hydroquinone treated samples to determine the methylation state of the nucleic acid being tested. Methylation-specific PCR provides an alternative method for the molecular testing of fragile X and other methylation associated disorders and can be used to identify affected and unaffected subjects even in the presence of mosaicism.

In bisulfite modification of the nucleic acid, unmethylated cytosine residues are converted to uracil, while methylated residues remain unconverted. The subsequent change in the sequence between affected and unaffected individuals after bisulfite treatment may be monitored by methylation-specific PCR. Methylation-specific PCR is a rapid assay that can be completed in two days and requires very little DNA for analysis, two important factors for prenatal diagnosis. Other advantages of the test are that it is non-radioactive, cost and labor efficient, making it amenable for routine diagnostics and screening studies. The methylation-specific PCR assay produces amplification specific for either presence or absence of methylation (or both), and thus provides an advantage over other screening methods where a positive result is dependent on an absence of product. The chemical modification of cytosine to uracil by bisulfite treatment provides a useful modification of traditional PCR techniques which eliminates the need for methylation specific restrictions enzymes.

Briefly described, in an embodiment of the present disclosure using methylation-specific PCR, after sodium bisulfite treatment, the sequence under investigation is then amplified by PCR with two sets of strand-specific primers (one set specific for the methylated DNA and the other specific for the umethylated DNA) to yield a pair of fragments, one from each strand, in which all uracil and thymine residues have been amplified as thymine and only 5-methylcytosine residues have been amplified as cytosine. The PCR products can be sequenced directly to provide a strand-specific average sequence for the population of molecules or can be cloned and sequenced to provide methylation maps of single DNA molecules. This assay requires only small quantities of DNA, is sensitive to 0.1% methylated alleles of a given CpG island locus, and can be performed on DNA extracted from paraffin-embedded samples. Methylation-specific PCR eliminates the false positive results inherent to previous PCR-based approaches which relied on differential restriction enzyme cleavage to distinguish methylated from unmethylated DNA. However, such methylation specific PCR assays do not allow for simultaneous quantitation of the ration of methylated to unmethylated DNA. Instead, in embodiments of the present disclosure, quantitative analysis can be performed after the completion of the PCR step by end-point analysis techniques, as discussed above.

As is known to those of skill in the art, PCR typically employs two primers that bind to a selected nucleic acid template. The primers are combined with the other PCR reagents under conditions that induce primer extension, e.g., with four different nucleoside triphosphates (or analogues thereof), an appropriate polymerase and an appropriate buffer (“buffer” includes pH, ionic strength, cofactors, etc.) at a suitable temperature. In some embodiments the primers are labeled primers (e.g., primers or short nucleotide sequences that are 5′ end-labeled with a reporter molecule (e.g., a fluorophore)) to allow for detection and quantification of bound probe after the PCR process. In exemplary embodiments of the present disclosure, PCR primers are prepared from the FMR1 gene and SRY gene (example primers are listed in the examples below) and PCR is carried out generally as described in the examples below.

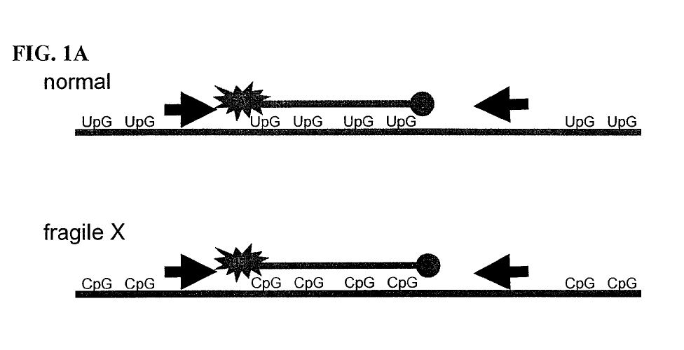

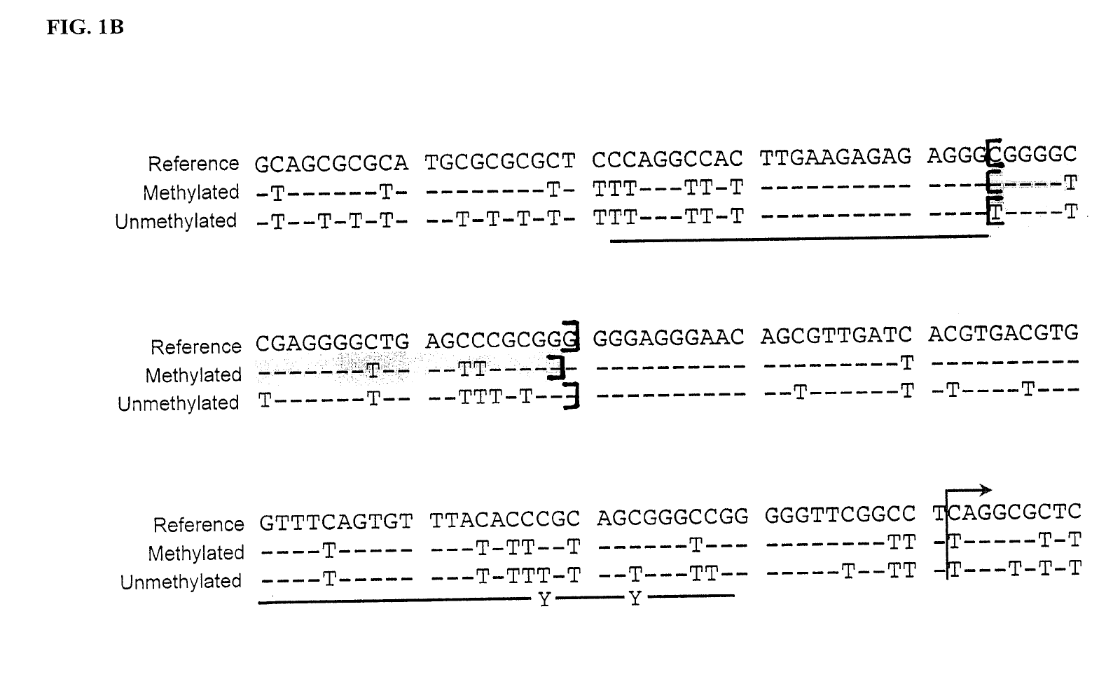

In another embodiment of the methods of present disclosure, a real time PCR detection method, such as Taqman, can be used to detect and quantify methylated and unmethylated FMR1 alleles after sodium bisulfite treatment in a single step. In the Taqman method the methylated and unmethylated strands are amplified by a single primer pair, avoiding the bias in PCR due preferential binding of oligonucleotides in PCR. This is accomplished by avoiding CpG dinucleotides in the primer binding sites. The distinction between the methylated and unmethylated strands is made by the Taqman probes targeting a specific CpG within the amplicon, one probe for the methylated strand and a second different (e.g., different fluorochrome) probe for the unmethylated strand. FIG. 1A illustrates the location of the differentially labeled probes in reference to a target sequence for a methylated and unmethylated (normal) version of a target sequence. Additional primers and probes can be included in the assay to amplify and identify other loci associated with other methylation-specific disorders as well as primers and probes to quantify the number of X and Y chromosomes present in the sample, as discussed in greater detail in the discussion and examples below. Not only is the Taqman method faster than the PCR and capillary electrophoresis method in that it detects and quantitates the amount of methylated and unmethylated DNA in a single step, it is also a more robust method for quantitation allowing the better distinction between fragile X females and normal females as well as determining the number of sex chromosomes present in a sample.

Preferably, the method of amplifying is by PCR, as described herein and as is commonly used by those of ordinary skill in the art. Alternative methods of amplification have been described and can also be employed as long as the methylated and non-methylated loci are similarly amplified by the alternative method.

Optionally, the methylation pattern of the nucleic acid can be confirmed by restriction enzyme digestion and Southern blot analysis. Examples of methylation sensitive restriction endonucleases that can be used to detect 5′CpG methylation include SmaI, SacII, EagI, MspI, HpaII, BstUI and BssHII, for example.

The methods of the present disclosure also provide enhanced diagnostic tests for fragile X syndrome that allow the diagnosis of both females and males with the disorder, as well as males mosaic for the disorder, by conducting DNA methylation analysis simultaneously with CGG repeat tract analysis in a single tube. In one example of an assay using endpoint analysis the methylation status of the FMR1gene is determined by quantitative methylation sensitive PCR with primers that target the FMR1 promoter. These primers are specific for sodium bisulfite treated DNA and are 5′ end-labeled with a fluorophore for detection by capillary electrophoresis as described herein. The FMR1 DNA methylation index is calculated by quantifying of peak areas for the methylated and unmethylated specific PCR products. In addition, a second set of primers is included to amplify the CGG repeat tract after sodium bisulfite conversion. These primers are 5′ end-labeled with a distinct fluorophore to distinguish these products from the methylation sensitive PCR product. The PCR product specific for the repeat tract can be analyzed to determine the size of the CGG repeats and determine if a CGG repeat expansion has occurred. The PCR reaction and thermal cycling parameters can be adjusted to amplify both normal and expanded alleles.

In a preferred embodiment, real-time PCR is used to detect and quantify methylated and unmethylated DNA in a single step (quantitative methylation sensitive PCR (Q-PCR)). Q-PCR involves the use of a single primer pair to amplify the target polynucleotide and a set of probes capable of distinguishing methylated from unmethylated DNA loci on the target polynucleotide (more than one primer pair and more than one set of probes may be used if more than one DNA loci is being analyzed in a single assay; for example testing for both fragile X and sex chromosome aneuploidies in a single assay). These methods are described in greater detail in the discussion and examples below. The analysis of the qualitative and quantitative results from either the kinetic or an endpoint analysis method is similar. Such analysis is described below.

For example, in a normal male only an unmethylated FMR1 gene and a normal repeat tract can be detected. In a fragile X male only a methylated FMR1 gene can be detected with absence of the CGG repeat in the normal range. A variation would be to determine the presence of abnormal size range CGG repeats, greater than 200 CGG repeats, by PCR that can also detect large alleles. Mosaic males, which may represent up to ¼ of all fragile X patients, can be detected as males with both methylated and unmethylated FMR1 alleles as well as the presence of CGG repeat tracts below the pathogenic threshold.

In a normal female both methylated and unmethylated FMR1 alleles can be detected in equal proportions. In addition, in many females two different sized repeat tract alleles can be detected, indicating the presence of two normal FMR1 alleles. A subset of females will have FMR1 alleles that have equal numbers of CGG repeats, thus only a single PCR product will be observed. However, these females would be scored as normal since they will have equal proportions of methylated and unmethylated FMR1 DNA. A variation would be to determine the presence of abnormal size range CGG repeats, greater than 200 CGG repeats, by PCR that can also detect large alleles. Fragile X females will have an altered ratio of methylated to unmethylated FMR1 DNA. Without any skewing of X chromosome inactivation the methylation index will be shifted from a 1:1 ratio to a 3:1 ratio. In addition, in these females only a single CGG repeat tract from the normal allele will be detected, since the expanded CGG repeat tract will not be able to be amplified by PCR.

A novel aspect of some embodiments of the present method(s) is that it can simultaneously identify individuals with sex chromosome abnormalities. In the context of population screening this becomes very important. In normal individuals females carry two X chromosomes and males carry one X and one Y chromosome. The FMR1 gene lies on the X chromosome and determination of the number of FMR1 genes present indicates the number of X chromosomes an individual carries. In addition to the X-linked FMR1 gene, a probe specific for the SRY gene located on the Y chromosome is also provided in the assay, allowing detection and quantitation of the male specific chromosome. The detection and quantitation of the Y chromosome, like FMR1 on the X chromosome, may be done by kinetic analysis (e.g., real-time PCR) or end-point analysis. The methods of the present disclosure detect and quantify the number of each of the sex chromosomes a person has leading to the identification of individuals with sex chromosome abnormalities.

For example, if only one X chromosome is detected the individual has Turner syndrome. If two X chromosomes and one Y chromosome are detected this individual has Klinefelter syndrome. There are several other abnormalities of sex chromosomes including 47,XXX syndrome, 47,XYY syndrome, etc. that can be detected by the present method. Collectively, these abnormalities are the most common chromosomal disorders in the population, with a frequency of 1 in 500. This is much greater than the estimated incidence of fragile X syndrome (somewhere between 1 in 3700 to 1 in 8900). Identification of these individuals, who are frequently not diagnosed at birth, would allow appropriate medical management for these individuals resulting in some cases in life saving treatments.

In addition, this population screening and/or diagnostic method can be applied to disorders that are associated with alterations in SNRPN DNA methylation at 15q11q13. These disorders include Prader-Willi, Angelman, and maternally inherited 15q11-13 duplications associated with autism. In normal individuals there is a 1:1 ratio of methylated and unmethylated SNRPN DNA. Absence of SNRPN methylation is indicative of Angelman Syndrome and detection of only SNRPN methylation is indicative of Prader-Willi syndrome. This segment of chromosome 15 is prone to both duplications and deletions due to the presence of repetitive sequences that flank this region. Maternally inherited duplications of this region are associated with autism and represent one of the most common known genetic causes of autism. All three of these disorders would be detected using the methodology of the present disclosure by detecting and quantifying methylated and unmethylated SNRPN DNA.

Silver Russell (SRS) and Beckwith-Wiedemann (BWS) syndromes are caused by defects in imprinted gene expression at chromosome 11p15. There are two domains that contain differentially methylated regions (DMRs) that control imprinted gene expression at 15p15. DMR1 is located within the telomeric domain that contains H19 and the IGF2 genes. DMR2 is located within the centromeric domain and contains several genes, including KCNQ1 and CDKN1C. Alterations in DNA methylation at both DMRs result in aberrant expression of these genes leading to disease. Hypermethylation of DMR1 is found in 2-13% of patients with BWS, a disorder characterized asymmetric overgrowth and cancer predisposition. Hypomethylation of DMR1 is found in approximately 35% of patients with SRS, a disorder characterized by asymmetric growth retardation. Quantitation of DNA methylation by this methodology at DMR1 would lead to the identification of individuals with these disorders. In addition, defects in DNA methylation of DMR2 in the centromeric domain are also associated with BWS. Approximately 60-70% of cases of BWS are found to have loss of methylation at DMR2.

In addition to the disorders described above, other disorders that are associated with defects in DNA methylation could also be identified using this methodology. Early identification of individuals with these disorders would allow initiation of medical treatments that would have a significant impact on the quality of life for these individuals and their families.

Thus, in yet further embodiments of the disclosure, a method is provided for screening male and female members of a population for fragile X syndrome, Prader-Willi syndrome, Angelman syndrome, maternally inherited 15q11-13 duplications associated with autism, SRS, and Beckwith-Wiedemann syndrome, and disorders resulting from sex chromosome aneuploidies. Additional embodiments provide an assay for simultaneously screening for on or more of the above disorders (and/or other disorders associated with abnormal methylation of DNA) in a single assay. Also provided is a kit containing the necessary elements for performing such an assay.

Embodiments of a kit according to the present disclosure include reagents for treating a tissue sample to modify unmethylated cytosine (e.g. a solution of sodium bisulfite and/or hydroquinone). In one embodiment of a kit according to the present disclosure, the kit includes the appropriate reagents, a set of primers specific for the unmethylated nucleic acid sequence(s) being screened and a set of primers specific for the methylated nucleic acid sequence(s) being screened for use in methylation-specific PCR. For instance, such a kit may contain primers specific for a methylated, sodium bisulfite/hydroquinone treated nucleic acid sequence from the promoter region of the FMR1 gene on the X chromosome and primers specific for an unmethylated, sodium bisulfite/hydroquinone treated nucleic acid sequence from the FMR1 gene of the X chromosome. In order to also determine the gender of the test subject and to screen for additional sex chromosome associated disorders, the kit may also contain primers specific for the SRY gene of the Y chromosome. In embodiments, the primer sets may also be labeled (“labeled primers”) to allow for quantification via end-point analysis (e.g. capillary electrophoresis).

In embodiments of a kit for use in quantitative methylation specific PCR methods of the present disclosure, the kit includes reagents for treating a tissue sample to modify unmethylated cytosine (e.g. a solution of sodium bisulfite and/or hydroquinone), a set of primers for amplifying both the methylated and unmethylated nucleic acid sequence being screened, and a set of probes for discriminating and providing for the real-time quantification of the methylated and unmethylated nucleic acid sequences being screened. For example, such a kit may contain a primers pair for amplifying the sodium bisulfite/hydroquinone treated nucleic acid sequence from the promoter region of the FMR1 gene on the X chromosome and probes capable of distinguishing the unmethylated from the methylated, sodium bisulfite/hydroquinone treated nucleic acid sequence from the FMR1 gene of the X chromosome. Such a kit also allows for determining the gender of the test subject and can screen for additional sex chromosome associated disorders by determining the number of X chromosomes present in the sample of an individual based on the ratio of methylated to unmethylated DNA from a loci on the X chromosome (e.g., FMR1); however, the kit may also contain primers and probes specific for the SRY gene of the Y chromosome for this purpose as well.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

### EXAMPLES

It should be emphasized that the above-described embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

### Example 1

### Population Screening for Fragile X Syndrome by Quantification of Methylated FMR1 DNA

### Materials and Methods

### Sodium Bisulfite Treatment of Genomic DNA

Sodium bisulfite treatment of genomic DNA was performed essentially as previously described (Kubota, T. et al., 1997 Methylation-specific PCR simplifies imprinting analysis. Nat Genet. 16:16-17, incorporated herein by reference). Briefly, 1 μg of genomic DNA isolated from peripheral blood lymphocytes was diluted in 25 μl dH20. The DNA was denatured by the addition of 2 μl of 2N NaOH to a final concentration of 0.2N NaOH, and incubated at 37° C. for 10 minutes. After denaturation, 15 μl of freshly prepared 10 mM hydroquinone (Sigma cat #H 9003) and 260 μl of 3.6M sodium bisulfite pH˜5.0 (Sigma cat #243973) were added and the reaction was layered with mineral oil and incubated 4-16 hrs at 54° C. A modification of the protocol for the Wizard SV Genomic DNA Clean-Up System (Promega A2361) was used to isolate the DNA after sodium bisulfite treatment. Briefly, 300 μl of a 1:1 mix of SV lysis buffer and 95% ethanol was mixed with the ˜300 μl of sodium bisulfite reaction. This mixture was transferred to a spin column and centrifuged 1 minute to bind the DNA to the resin within the column. The sample was washed two times with 600 μl of SV Wash Buffer, centrifuging 1 minute for each wash. The column was centrifuged one more time without wash buffer to remove residual buffer and transferred to a new 1.5 ml microfuge tube. The DNA was eluted from the column by adding 50 μL of H₂O followed by centrifugation for 1 minute. The DNA eluted from the column was desulfonated by the addition of 5.5 μl 3N NaOH and incubation at room temperature for 5 minutes. The DNA was ethanol precipitated, washed 1 time with 75% ethanol and suspended in 50 μl of EB (10 mM Tris HCl pH 8.0).

### Sodium Bisulfite Treatment of Dried Blood Spots

Two 3 mm dried blood spot punches were immersed in 30 μl of 1% SDS and heated to 97° C. for 10 minutes to release the DNA. After heating, 6.75 μl of the extract was transferred to a fresh tube and the DNA denatured by the addition of 0.75 μl of 2N NaOH. After denaturation 3.75 μl of freshly prepared 10 mM hydroquinone and 65.25 μl of 3.6 M NaHSO₃ was added and the reaction incubated at 54° C. for 4-16 hours. After incubation the DNA was purified with the Wizard SV-96 DNA Clean-up System. To desulfonate the DNA NaOH was added to a final concentration of 0.1 N and incubation at 37° C. After incubation the sample was neutralized with HCl and buffered by the addition of Tris HCl (pH 8.0) to a final concentration of 10 mM. All of the steps of sodium bisulfite treatment, DNA clean-up, and desulfonation were performed using the BiomekFX robotic platform.

### Real-Time Taqman Methylation Sensitive PCR for FMR1 Methylation

Primer and probe design for FMR1: All primers for the Taqman MSP were synthesized by Integrated DNA Technologies. The amplification primers used in the real-time Taqman methylation sensitive PCR reaction were designed to avoid CpG dinucleotides in the sense strand within the promoter of the FMR1 gene. The FMR1 amplification primers are: FMR1F 5′-GYGTTTTTAGGTTATTTGAAGAGAGAGGG-3′ (SEQ ID NO: 1) and FMR1R 5′-CRACCCRCTACRAATATAAACACTAAAACC-3′ (SEQ ID NO: 2). The Taqman probes FMRM2 (methylated DNA specific probe) and FMRU3 (unmethylated FMR1 DNA specific probe) target a sequence from positions −97 to −72, relative to transcription initiation site for FMR1. FMRM2 (5′-CGGGGTCGAGGGGTTGAGTTCGCG-3′) (SEQ ID NO: 3) is 5′ end-labeled with FAM and is quenched by the addition of Black Hole Quencher 1 to the 3′ end of the oligonucleotide. FMRU3 (5′-TGGGGTTGAGGGGTTGAGTTTGTGGG-3′) (SEQ ID NO: 4) is 5′ end-labeled with HEX and quenched by the addition of Black Hole Quencher 1 to the 3′ end of the oligonucleotide. FIG. 1B illustrates the location of the primer and probe sequences in the promoter region of the FMR1 gene.

Real-time Taqman Methylation Sensitive PCR set-up: The methylation status of the FMR1 promoter was assessed using 16 ng of sodium bisulfite treated genomic DNA suspended in 5 μl of EB. The PCR was performed in a 25 μl reaction with 1× Invitrogen PCR buffer (20 mM Tris-HCl pH 8.4 and 50 mM KCl), 2.5 mM MgCl₂, 250 μM dNTPs, 2.5 pmol/μl of each amplification primer, 0.15 pmol/μl of each Taqman probe, and 8 units of Invitrogen Platinum Taq (catalog number 10966). The PCR cycling parameters using a BioRad iQ5 Light-Cycler were: initial denaturation at 95° C. for 3 minutes followed by (95° C. for 10 seconds, 67° C. for 30 seconds and 72° C. for 30 seconds) repeated 40 times. In parallel with each set of reactions a standard curve was generated using a sodium bisulfite treated genomic DNA sample from an unaffected female. The range of DNA amount used in the standard curve was from 1 ng to 64 ng. The crossing-threshold was plotted against the amount of input DNA (expressed as copies of FMR1), and the amount of methylated and unmethylated FMR1 DNA was estimated by interpolation on this curve.

### Results

### Design of the Quantitative Methylation Sensitive PCR (Q-MSP) Assay

The FMR1 promoter was chosen as a target for the DNA methylation analysis. Amplification primers and Taqman probes described above were designed to assess a DNA sequence immediately upstream of the transcription initiation site, from positions −97 to −72, on the sense strand in the FMR1 promoter. Extensive methylation is found throughout the entire FMR1 promoter in males with fragile X syndrome. Unlike conventional methylation sensitive PCR methods, the Taqman probes, and not the amplification primers, are used to discriminate between methylated and umethylated DNAs after sodium bisulfite treatment. The amplification primers were designed to avoid CpG dinucleotides, to minimize any bias in amplification of methylated and unmethylated DNAs (FIG. 1B). The Taqman probes span 4 CpG dinucleotides and target the two Sp1 sites located in the FMR1 promoter. The Taqman probe designed to hybridize to methylated FMR1 DNA after bisulfite treatment is labeled with FAM. As illustrated in FIG. 1A and FIG. 1B, the Taqman probe designed to hybridize unmethylated FMR1 DNA is labeled with HEX. Thus, this method allows the independent assessment of the amount of methylated and unmethylated FMR1 DNA simultaneously within a single sample.

A standard curve using DNA from a normal female is generated each time an assay is performed to quantify the amounts of methylated and unmethylated FMR1. Normal females will have equivalent amounts of methylated and unmethylated FMR1 DNA, due to random X inactivation. Sodium bisulfite DNA is titrated from 64 ng to 1 ng to produce the standard curve. Representative amplification plots are shown in FIG. 2 for both methylated (the FAM labeled Taqman probe) and unmethylated (the HEX labeled Taqman probe) DNA. The crossing threshold for each known concentration is plotted against the amount of input DNA. The correlation coefficients (R²) for each of these curves typically exceed 0.95. The amount of methylated and unmethylated FMR1 DNA present in the unknown samples is determined by interpolation.

### Detection and Quantification of Methylated FMR1 DNA in Males

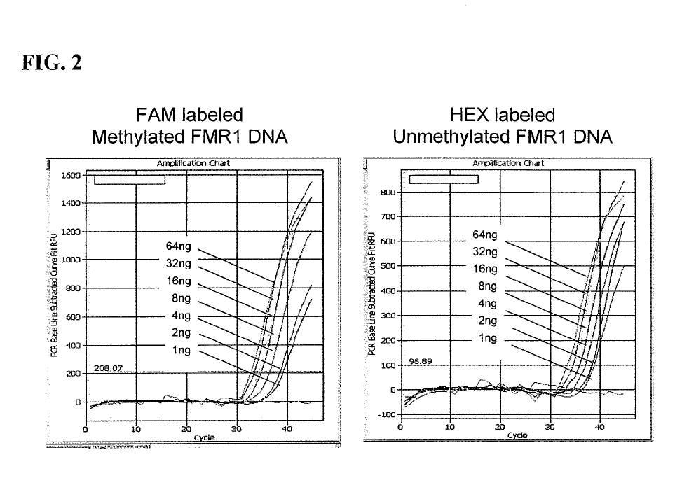

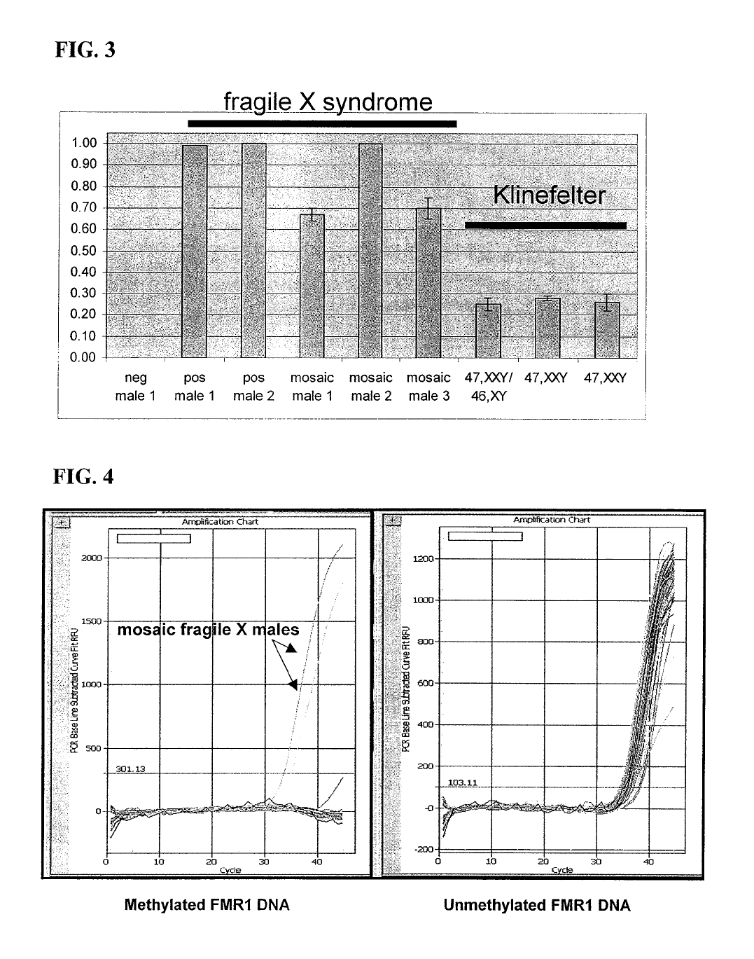

The presence of any methylated FMR1 DNA in a male is indicative of an abnormality. In addition to fragile X syndrome, methylated FMR1 DNA found in a male sample would be consistent with Klinefelter Syndrome (47,XXY) or 46,XX sex reversal. After quantification, the amount of methylated FMR1 DNA can be expressed as a methylation index (MI), defined as the amount of methylated FMR1 DNA divided by methylated plus unmethylated FMR1 DNA. Since a normal male will not have any methylated FMR1 DNA, they will always have a MI of zero. A male with fragile X syndrome, Klinefelter syndrome (or one of its variants), or a 46,XX male will carry methylated FMR1 DNA and will have a MI greater than zero. FIG. 3 illustrates representative results obtained from screening a negative male, mosaic and full mutation fragile X males, and mosaic and full Klinefelter males. Q-MSP can readily detect FMR1 DNA methylation in males whether due to CGG repeat expansion or the presence of more than one X chromosome.

### Adaptation of the Q-MSP Assay to a High-Throughput Format

For population screening, Q-MSP would need to be adapted to a high-throughput format. The sodium bisulfite treatment can also be performed in 96-well plates using dried blood spots as starting material as described above. The first step in the procedure is to boil the dried blood sample in 1% SDS for 10 minutes to release the DNA. This crude extract is then used directly for sodium bisulfite treatment, essentially following the same procedures used for isolated genomic DNA. After sodium bisulfite treatment the DNA is purified using the Wizard SV-96 DNA Clean-up System. After purification the sodium bisulfite treated DNA is desulfonated prior to PCR. In conventional protocols, this is accomplished by a brief treatment with NaOH followed by ethanol precipitation to clean-up the DNA a second time. For dried blood spot methylation analysis the ethanol precipitation was replaced with a simple neutralization step followed by buffering of the sample in 10 mM Tris (HCl). This modification simplifies the procedure and makes it more amenable for high-throughput processing.

88 male dried blood samples were screened for methylated and unmethylated FMR1 DNA using this procedure (FIG. 4). Two mosaic male fragile X samples were introduced into this cohort of samples, their location was blinded. The other 86 samples were from normal males. Eight wells were reserved for the standard controls. The two mosaic fragile X males were easily identified among the 86 negative males by the detection of methylated FMR1 DNA (FIG. 4). These results demonstrate the feasibility of this approach for large-scale population screening.

Q-MSP is very sensitive and in experiments where genomic DNA from a fragile X male is mixed with DNA from a normal male, approximately 1% methylated FMR1 DNA is detectable in a background of 99% unmethylated FMR1 DNA (data not shown). The sensitivity of the assay will allow the detection of mosaic fragile X males and mosaic Klinefelter syndrome relatively easily. In a population screen the vast majority of males would test negative for the presence of methylated FMR1 DNA. Given the sensitivity of the assay, it was reasoned that all male samples could be pooled together in a single tube and assessed for FMR1 DNA methylation for all samples simultaneously. If FMR1 DNA methylation is detected, then each dried blood spot can then be tested individually to identify the positive sample within that set.

To test this idea, one dried blood spot from a mosaic fragile X patient was added to 95 dried blood spots from normal males in a single tube. The procedure was performed as described above. A set of samples from 96 normal males served as a control. Methylated FMR1 DNA from the single mosaic fragile X male sample was able to be detected from among 95 normal male dried blood spots (FIG. 5). These results again demonstrate the sensitivity of this method and allow for the possibility of simultaneously screening up to 100 males at a time for methylated FMR1 DNA, which will greatly improve the efficiency and lower the cost of the assay.

### Quantification of Methylated and Unmethylated FMR1 DNA Females

A normal female will possess equivalent amounts of methylated and unmethylated FMR1 DNA due to random X inactivation, resulting in a MI of about 0.50. In fragile X females, without skewing of X inactivation, there would be about a 3 to 1 ratio of methylated to unmethylated FMR1 DNA, resulting in a MI of about 0.75. Skewing of X inactivation could alter the MI from 0.50 (completely favorably skewed—i.e., the inactive X always carries the mutated FMR1 gene) to 1.00 (completely unfavorably skewed—i.e., the active X always carries the mutant FMR1 gene). A female with an elevated MI would be at an increased risk of carrying an expanded, abnormally methylated FMR1 allele.

The MIs for a series of normal and fragile X females were calculated to determine if it was possible to distinguish between normal female FMR1 DNA methylation and fragile X female FMR1 DNA methylation (FIG. 6). The mean MI for 13 normal females was 0.59 with a SD of 0.07. The slight variation from the predicted 0.50 is probably due to some low level cross hybridization between the Taqman probes. Assuming a normal distribution, from this data set it can be estimated that 95% of normal females will have a MI of between 0.45 and 0.73, with 2.5% having a MI below 0.45 and 2.5% having a MI above 0.73. The mean MI for a cohort of 33 full mutation females is 0.80 with a SD of 0.09. Thus, the MI index is significantly elevated among full mutation carrier females when compared to normal females (P=5.4×10⁹; alpha=0.05). Twenty-seven out of the thirty-three fragile X females have a MI of greater than 2 SD above the mean. Using 2 SD above the mean as a cutoff (0.74 and greater), 82% of full mutation carrier females will have an elevated MI with only 2.5% of normal females having a MI above that cut-off (false positives).

Approximately half of all females who carry full mutation FMR1 alleles will be affected with fragile X syndrome. The reduced penetrance of fragile X syndrome in females is thought to be due to skewing of X inactivation, especially in the brain. In this cohort of full mutation carrier females 19 were classified as affected, 5 were classified as unaffected, and for 9 of the females phenotypic information was not available. Of the 19 classified as affected, 15 had a MI of 0.74 or greater (79%). Of the 5 classified as unaffected, 4 had a MI of greater than 0.74 with one having a MI below 0.73 (80%). Therefore, the probability that an unaffected full mutation carrier female will have an elevated MI is the same as the probability that an affected full mutation carrier will have an elevated MI.

### Discussion

This sensitivity of the Q-MSP assays of the present disclosure not only allow for the detection in male subjects of low level fragile X and Klinefelter mosaics, but also allows for the pooling of male samples into large groups permitting the simultaneous screening of up to 100 males for methylated FMR1 DNA. The distinction between these disorders could be made by additional testing of either the DNA from the dried blood spot card or from follow-up testing of patients in a clinic.

Q-MSP could also be used to screen for fragile X full mutation, as well as sex chromosome aneuploidies, in females. Detection of only unmethylated FMR1 DNA in a female, a typical male pattern, would be consistent with a diagnosis of Turner syndrome, 46,XY sex reversal, or severe androgen insensitivity resulting in feminization of a 46,XY male. An increase in FMR1 DNA methylation would be consistent with either fragile X syndrome, 47,XXX or other sex chromosome aneuploidies in which more than two X chromosomes are present. As in males, the distinction between these possibilities could be made after follow up testing.

This methodology has many features attractive to newborn screening programs across the U.S. Identification of fragile X syndrome and the sex chromosome aneuploidies in the neonatal period would allow for early intervention for these children improving the outcome for individuals. Identification of these children in a newborn screen would also prevent the stress and anxiety, in addition to the monetary costs, parents undertake in their “diagnostic odyssey”. Currently, it takes 3-4 years to obtain a diagnosis of fragile X syndrome. Identification of children with the full mutation would lead to identification of permutation and clinically unrecognized full mutation mothers. These women could be counseled appropriately about the risks of fragile X syndrome in future pregnancies. In addition, these women, as well as members of their family, could be counseled about their risks of premature ovarian failure and FXTAS.

There are several advantages of Q-MSP over sizing the CGG repeat tract for population screening. First, there is direct detection of the mutation (FMR1 methylation) that causes fragile X syndrome and a positive result does not have to be inferred by the absence of a PCR product, as is the case for CGG repeat sizing. Second, mosaics are easily detected by Q-MSP, whereas there is a potential to miss a mosaic by sizing of the CGG repeat tract if there are both normal size and expanded CGG alleles present. If used for newborn screening, it is estimated that FMR1 methylation analysis would lead to the identification of an abnormality in 1 out of every 735 births. Thus, Q-MSP for FMR1 DNA methylation can be used for the identification of both fragile X syndrome and sex chromosome abnormalities in a single assay.

### Example 2

### Real-Time Taqman Methylation Sensitive PCR for Angelman, Prader-Willi, 15q11-13 Maternally Inherited Duplication: Quantification of SNRPN Methylation

### Materials and Methods

Sodium bisulfite treatment of genomic DNA and/or dried bloodspots was performed as described in Example 1.

Primer and probe design for SNRPN promoter: The amplification primers target the sense strand of the SNRPN promoter. The SNRPN amplification primers were: SNRPNF-GGAGGGAGTTGGGATTTTTG (SEQ ID NO: 5) and SNRPNR-ACAAACTTCRCACACATCCC. (SEQ ID NO: 6). The Taqman probes that target the SNRPN promoter are: SNRPNM-TGCGGTAAATAAGTACGTTTGCGCGGTCGTAG (SEQ ID NO: 7) and SNRPNU-TGTGGTAAATAAGTATGTTTGTGTGGTTGTAGAGGTAGGTTGGTG. (SEQ ID NO: 8). The Taqman probe specific for the methylated DNA is labeled with FAM and the unmethylated DNA specific probe is labeled with HEX. Both probes were quenched by the addition of Black Hole Quencher 1 to the 3′ end of the oligonucleotide.

Real-time Tagman Methylation Sensitive PCR set-up: The methylation status of the SNRPN promoter was assessed using sodium bisulfite treated genomic DNA suspended in 5 μl of EB. The PCR was performed in a 25 μl reaction with 1× Invitrogen PCR buffer (20 mM Tris-HCl pH8.4 and 50 mM KCl), 2.5 mM MgCl₂, 250 μM dNTPs, 2.5 pmol/μl of each amplification primer, 0.15 pmol/μl of each Taqman probe, and 8 units of Invitrogen Platinum Taq (catalog number 10966). The PCR cycling parameters using a BioRad iQ5 Light-Cycler were: initial denaturation at 95° C. for 3 minutes followed by (95° C. for 10 seconds, 52° C. for 30 seconds, and 72° C. for 30 seconds), repeated 40 times. In parallel with each set of reactions a standard curve was generated using a sodium bisulfite treated genomic DNA sample from an individual. The range of DNA amount used in the standard curve was from 1 ng to 64 ng. The crossing-threshold is plotted against the amount of input DNA (expressed as copies of SNRPN) and the amount of methylated and unmethylated SNRPN DNA estimated by interpolation on this curve.

### Results

SNRPN is an imprinted gene that is methylated on the maternal allele and unmethylated on the paternal allele. Correct imprinted expression is necessary for normal physiological and cognitive development. A normal individual will have a 1:1 ratio of methylated to unmethylated SNRPN DNA (a methylation index of about 0.5), since they inherit one copy of SNRPN from their mother and one copy from their father. An individual with Angelman Syndrome will have loss of methylated SNRPN DNA (a methylation index of about 0.00), whereas an individual with Prader-Willi Syndrome will have loss of unmethylated SNRPN DNA (a methylation index of about 1.0). An individual with a duplication of 15q11-13 will have a two to one ratio of methylated to unmethylated SNRPN DNA (a methylation index of about 0.67). An individual with a triplication of maternally inherited 15q11-13 will have a 3:1 ratio of methylated to unmethylated SNRPN DNA (a methylation index of about 0.67).

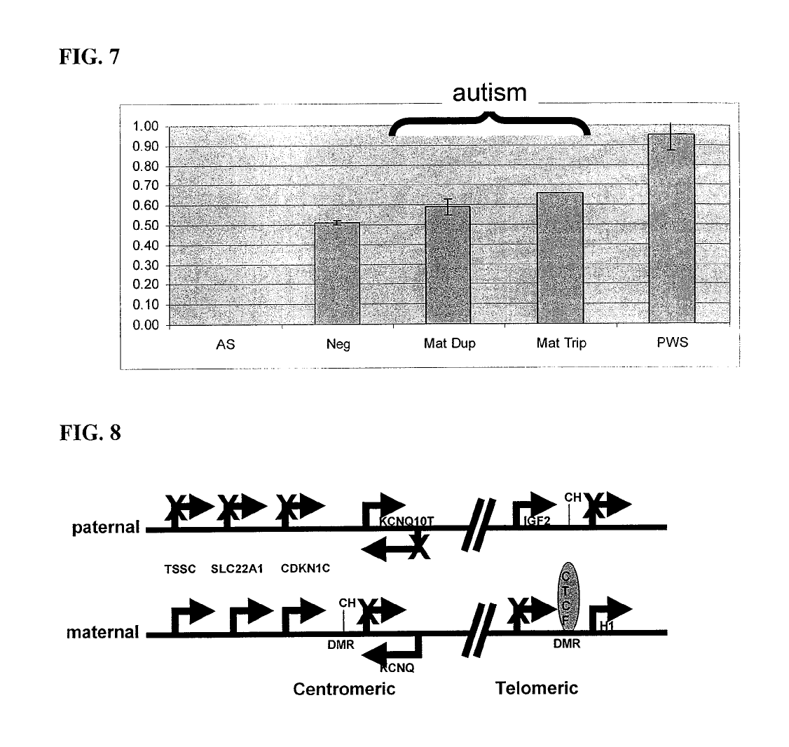

As with the quantification of methylated and unmethylated FMR1 DNA, quantification of methylated and unmethylated SNRPN DNA was done interpolation of the crossing threshold of a sample on a standard curve generated by titration of a sample from an unaffected individual. The methylation indices for an unaffected individual, an individual with Angelman syndrome (due to deletion of the maternal copy of 15q11-13), a maternally inherited duplication of 15q11-13, a maternally inherited triplication of 15q11-13, and an individual with a Prader-Willi Syndrome (due to the deletion of the paternal copy of 15q11-13) are shown in FIG. 7. These results indicate that this method can detect and quantify the amount of methylated and unmethylated SNRPN DNA carried by an individual, allowing the screening for these disorders.

### Example 3

### Real-Time Taqman Methylation Sensitive PCR for Beckwith-Wiedemann and Silver-Russell Syndromes: Quantification of H19 (DMR1) and KCNQ10T1 (DMR2) Methylation

### Materials and Methods

Sodium bisulfite treatment of genomic DNA and/or dried bloodspots was performed as described in Example 1.

Primer and probe design for DMR2—the KCNQ10T1 promoter: All primers for the Taqman MSP were synthesized by Integrated DNA Technologies. The amplification primers used in the real-time Taqman methylation sensitive PCR reaction were designed to avoid CpG dinucleotides in sense strand in the promoter of the KCNQ10T1 gene, which is antisense to the KCNQ1 gene. The KCNQ10T1 amplification primers are: Lit1F-GTTTAATTAGTAGGTGGGGGG (SEQ ID NO: 9) and Lit1R-CCTAACAAAATCTTACTAAAAAACTCC (SEQ ID NO: 10). The Taqman probes target the sequence −6 to −34 relative to transcription initiation of KCNQ10T1 (GenBank AJ006345; positions 255,041-255,069 on the anti-sense strand). The Taqman probe sequences are: Lit1-M-CGGCGGGGGTAGTCGGAGCG (SEQ ID NO: 11) and Lit1-U TGGTGGGGGTAGTTGGAGTGTTGTTGTAG (SEQ. ID NO: 12) (underlined sequences indicate CpG dinucleotides used to discriminate between methylated and unmethylated DNA). The methylated DNA specific probe is 5′ end-labeled with FAM and quenched by the addition of Black Hole Quencher 1 to the 3′ end of the oligonucleotide. The unmethylated DNA specific probe was 5′ end-labeled with HEX and quenched by the addition of Black Hole Quencher 1 to the 3′ end of the oligonucleotide.

Primer and probe design for DMR1-CTCF binding site 6: The amplification primers target the sense strand and flank the CTCF binding site 6, located upstream of the H19 gene. The CTCF-6 amplification primers were: CTCF6F-GTATAGTATATGGGTATTTTTGGAGG (SEQ ID NO: 13) and CTCF6R-CCCAATTAAAACRAACTCRAACTATAAT (SEQ ID NO: 14). The Taqman probes target the core sequence of the CTCF binding site 6 (GeriBank AC087017; positions 6183-6206). The probe sequences were: CTCF6M-AAGTGGTCGCGCGGCGGTAGTGTA (SEQ ID NO: 15) and CTCF6U-TGGAAGTGGTTGTGTGGTGGTAGTGTAGG (SEQ ID NO: 16). As with the KCNQ10T1 promoter, the Taqman probe specific for the methylated DNA is labeled with FAM and the unmethylated DNA specific probe is labeled with HEX. Both probes were quenched by the addition of Black Hole Quencher 1 to the 3′ end of the oligonucleotide.

Real-time Taqman Methylation Sensitive PCR set-up: In separate reactions the methylation status of the KCNQ10T1 promoter and CTCF binding site 6 were assessed using 16 ng of sodium bisulfite treated genomic DNA suspended in 5111 of EB. The PCR was performed in a 25 μl reaction with 1× Invitrogen PCR buffer (20 mM Tris-HCl pH 8.4 and 50 mM KCl), 1 mM MgCl₂, 250 μM dNTPs, 2.5 pmol/μl of each amplification primer, 0.15 pmol/μl of each Taqman probe, and 8 units of Invitrogen Platinum Taq (catalog number 10966). The PCR cycling parameters using a BioRad iQ5 Light-Cycler were: initial denaturation at 95° C. for 3 minutes followed by (95° C. for 10 seconds, 52° C. for 30 seconds and 72° C. for 30 seconds) repeated 40 times. In parallel with each set of reactions a standard curve was generated using a sodium bisulfite treated genomic DNA sample from an unaffected individual. The range of DNA amount used in the standard curve was from 1 ng to 64 ng.

Data analysis: The amount of methylated and unmethylated was calculated for each sample, which was assayed in triplicate, by interpolation on the standard curve that was ran in parallel with the unknown samples. The methylation index (MI) was determined by dividing the amount of methylated DNA by the amount of total DNA (methylated plus unmethylated DNA). The mean of the three assays was used to calculate the MI for that patient. Testing a series of unaffected individuals, which were also assessed in triplicate, generated a normal range. The mean and standard deviation was calculated for this set of samples and as with the Southern method² the normal range was defined as the mean of this set of normals±two standard deviations

### Results

Defects in imprinted gene expression at 11p15 are associated with Beckwith-Wiedemann Syndrome (BWS; for review; see reference 1). Greater than 70% of cases are found to have alterations in DNA methylation at two distinct differentially methylated regions (DMRs) at 11p15. DMR1 is located within the telomeric domain and controls the reciprocal imprinted expression of IGF2 and H19. The centromeric domain contains the KCNQ1, CDKN1C, SLC22A1L and the TSSC3 genes. The imprinted expression of these genes is controlled by KCNQ10T1, located on the antisense strand in intron 10 of the KCNQ1 gene. The transcription of KCNQ10T1 is regulated by methylation at DMR2 (FIG. 8).

IGF2 is expressed exclusively from the paternal allele and is silent on the maternal allele. H19 has the opposite imprint, expressed only from the maternal allele and silent on the paternal allele (FIG. 8). DNA methylation plays a critical role in the establishment of the correct chromatin structure for the imprinted expression of these two genes. In part, the CTCF chromatin insulator, which is sensitive to DNA methylation, mediates formation of the correct chromatin structure, and thus CTCF will only interact with unmethylated DNA. There are seven binding sites for CTCF, located between the IGF2 and H19 genes. In normal individuals the CTCF sites are methylated on the paternal allele, along with the H19 promoter, and unmethylated on the maternal allele.

CTCF binding to its cognate sites on the unmethylated maternal allele allows the formation of a chromatin loop that results in the expression of H19 and silences IGF2. Gaston et al., Analysis of the methylation status of the KCNQ10T and H19 genes in leukocyte DNA for the diagnosis and prognosis of Beckwith-Wiedemann syndrome. Eur J. Hum. Genet. 2001; 6:409-418, (incorporated herein by reference) found that out of 97 patients, 58 (60%) displayed hypomethylation of DMR2. Of the remaining 39 patients, 13 displayed hypermethylation of DMR1. Altogether, 71 out of 97 patients (˜73%) of patients displayed hypomethylation of DMR2 or hypermethylation of DMR1. The hypermethylation of H19 results in an increase in expression of IGF2 leading to the overgrowth phenotype. It is important to note that only methylation at the H19 promoter, and not at the CTCF binding sites, is assessed in clinical laboratory testing.

Recently, the opposite methylation defect has been identified in patients diagnosed with Silver-Russell Syndrome (SRS). In some SRS patients there is hypomethylation of the CTCF binding sites resulting in a decrease in IGF2 expression resulting in the growth retardation phenotype. In SRS hypomethylation of H19 is found in approximately 35% of patients clinically diagnosed with the disease.

Defects in DNA methylation are also found at the centromeric DMR2 resulting in BWS. Unlike DMR1, defects in DNA methylation at DMR2 have not been associated with SRS. The most common DNA methylation abnormality found in BWS is hypomethylation of the KCNQ10T1 promoter, which results in the aberrant expression of the KCNQ10T1 anti-sense transcript leading to the silencing of the genes located within DMR2. Approximately 60% of the cases of BWS are associated with loss of KCNQ10T1 DNA methylation. Paternal UPD is associated with about 10-20% cases, also resulting in loss of expression of the KCNQ1, CDKN1C, SLC22A1L, and TSSC3. Therefore, in about 70-80% of the cases of BWS there is partial or complete loss of KCNQ10T1 DNA methylation, due either to hypomethylation of the maternal KCNQ10T1 allele or due to segmental paternal UPD for chromosome 11.

### Assessment of DNA Methylation of DMR2 in Normal Individuals

Initially, an assay for BWS was developed that would have the highest yield, accounting for 60-80% of cases, methylation analysis of DMR2 and the KCNQ10T1 promoter. The amplification primers that target the sense strand of DMR2 were designed to avoid CpG dinucleotides, eliminating any bias in amplification between methylated and unmethylated templates. The Taqman probes target the KCNQ10T1 promoter at positions −15 to −34 for the methylated DNA specific probe and positions −6 to −34 for the unmethylated DNA specific probe relative to the transcription initiation site. The probes were labeled with FAM for methylated DNA and with HEX for unmethylated DNA.

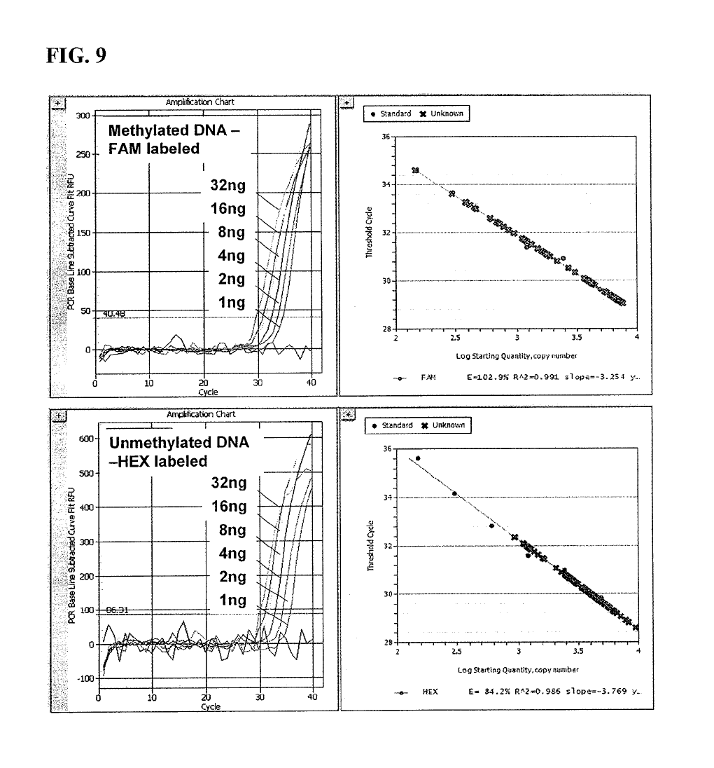

A standard curve was generated by titrating sodium bisulfite treated DNA, from 1 ng to 64 ng, from an unaffected individual. The curve was then used to determine the values of the unknown samples. A representative example of the amplification plots and the resulting standard curves for both methylated DMR2 DNA and unmethylated DMR2 DNA are shown in FIG. 9. The crossing threshold for each individual amplification correlates with the amount of input DNA allowing the generation of standard curves with correlation coefficients (R²) consistently greater than 0.95.

DMR2 DNA Methylation in Patients Diagnosed with BWS

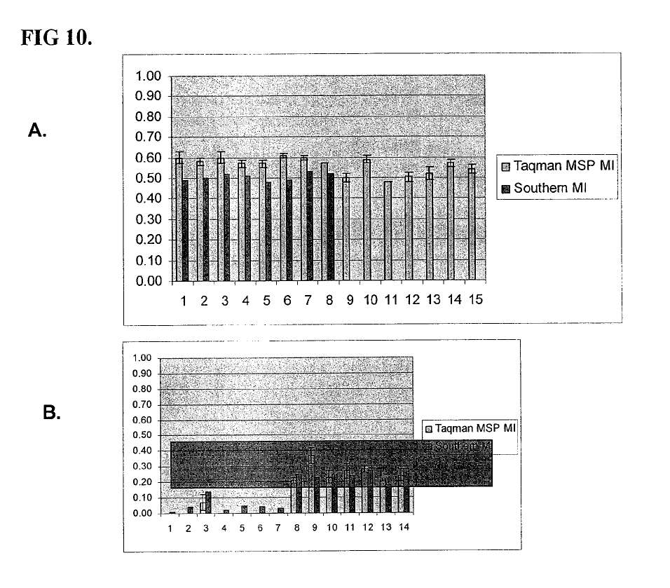

A series of unaffected individuals were tested to establish a normal range of DNA methylation detected in the assay (FIG. 10A). In Southern analysis for BWS the normal range is defined as the mean±two standard deviations (SD). For clinical testing by Southern analysis the normal range is accepted to be 0.40-0.60. An individual with a MI two SD below that mean is considered to have loss of methylation of DMR2, consistent with a diagnosis of BWS. FIG. 10A shows that the MIs for 15 unaffected individuals assessed for DNA methylation changes at DMR2. Eight of these individuals were also assessed by Southern analysis, shown as the as the dark gray bars for samples 1-8. In total, 92 DNA samples from individuals not diagnosed with BWS were assessed for methylation at DMR2. The average MI was 0.52 with a SD of 0.06 (data not shown).

DMR2 methylation was also assessed in a series of BWS patients who previously were identified to have loss of methylation by Southern analysis. In FIG. 10B the MIs from Taqman MSP were compared to Southern analysis in 14 patients with BWS. In all the patients with complete (or near complete) loss of DMR2 methylation (samples 1-7), as well as in samples with partial loss of DNA methylation, patients (samples 8-14), the Taqman MSP detected loss of KCNQ10T1 methylation that was similar to the MIs determined by Southern analysis. All the Taqman MSP analysis was done blinded to knowledge of the Southern analysis results.

### DNA Methylation of CTCF Binding 6 Upstream of H19

One advantage of Taqman MSP is that primers and probes can be designed to assess DNA methylation at any site in the genome. Since alterations in DNA methylation in DMR1 are also implicated in BWS and SRS, primers were designed to assess DNA methylation at this locus as well. Approximately 33% of patients with BWS who test negative for loss of KCNQ10T1 methylation have hypermethylation of the H19 promoter. In addition, 35% of patients clinically diagnosed with SRS have hypomethylation at H19. Instead of targeting the H19 promoter for methylation analysis Taqman probes and amplification primers were designed that directly target the CTCF binding site 6. This site is located at positions −2123 to −2137 relative to the H19 gene transcription start site. In the Southern analysis used in clinical testing a SmaI site, located at position −15 relative to transcription initiation, is targeted for DNA methylation analysis.

In FIG. 1A a series of 11 controls were tested to establish a normal range for the methylation index at DMR1. The mean from these 11 controls was 0.52 with a standard deviation of 0.05 resulting in a normal range from 0.42-0.62. This compares favorably with a mean of 0.51 and a normal range of 0.43-0.59 for Southern analysis (P. Lapunzina, unpublished data). In total, 91 DNA samples from normal individuals were assessed for methylation at DMR1. The average MI was 0.49 with a SD of 0.08 (data not shown).

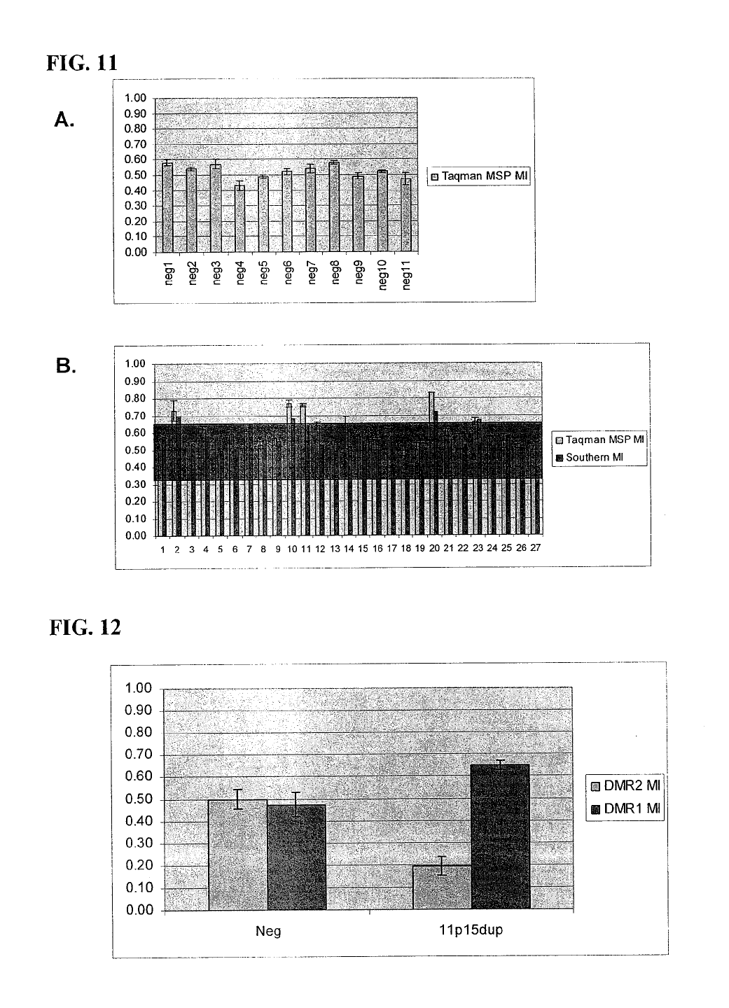

In addition, 27 BWS patients who were previously tested by Southern analysis for methylation defects at DMR1 and DMR2 were tested by Taqman MSP. All of the Taqman MSP results for DMR2 methylation analysis were completely concordant with the Southern analysis results (data not shown). The DMR1 analysis results are shown in FIG. 11B. As with DMR2 methylation analysis, the results of the DMR1 Southern analysis were blinded.

Of the five patients who scored positive for hypermethylation at CTCF6 (MI>0.65), all five scored positive for H19 promoter hypermethylation by Southern (MI>0.60), demonstrating the concordance between the two methods. One patient, sample 7, had a high normal Taqman MSP MI of 0.62 and a Southern MI of 0.65. This patient, therefore, had a Taqman MI slightly below the cut-off of 0.65, but scored positive by Southern with a MI slightly above the cut-off of 0.60. Interestingly, this patient also had hypomethylation of DMR2 both by Taqman MSP and Southern analysis (data not shown) and has a duplication of 11p15.

### Determining the Parent of Origin of a Cytogenetically Identified 11p15 Duplication

A duplication of 11p15 was identified by chromosome analysis in an infant with some of the clinical features of BWS, but without a clear clinical diagnosis of the disease. The combined assay of DMR1 and DMR2 methylation analysis was used to determine the parent of origin of this duplicated chromosome 11p15 segment. If the duplication was paternal in origin, as is found in some cases of BWS, a decrease in the MI for DMR2 from ˜0.5 to ˜0.33 would be expected since there would be two unmethylated paternal copies of DMR2 for every methylated maternal copy of DMR2. At the same time an increase in the MI at DMR1 from ˜0.5 to ˜0.66 would be seen. In this case there would two methylated paternal copies of the DMR1 for every unmethylated maternal copy of DMR1. As seen in FIG. 12 there is a shift in the MIs predicted for a paternal origin of this duplication indicating that the child has a methylation profile consistent with a diagnosis of BWS in this patient.

There are several advantages of the Taqman MSP over Southern analysis. Only 1 μg of DNA is needed for the Taqman MSP method as opposed to up to 10 μg required for Southern analysis. A second advantage is that the assay can be completed in 2 days versus the typical 1 week required for Southern analysis resulting in faster turn-around-time. A third advantage is that the Taqman MSP is less labor intensive and therefore more cost effective. A fourth advantage is that the assay is amenable to high-throughput analyses. For example, with a 96-well format instrument such as the iQ5 iCycler (BioRad) twenty-eight samples can be assayed simultaneously in triplicate, along with the controls for generating the standard curve. Finally, with this method any site in the genome can be targeted for DNA methylation analysis. By simply designing amplification primers that avoid CpG dinucleotides and Taqman probes that discriminate between methylated and unmethylated DNA after sodium bisulfite conversion any CpG island in the human genome can be assessed for alterations in DNA methylation. Thus, clinical laboratory testing using this approach could be readily developed for all human epigenetic disorders.

### Example 4

### Methylation Specific PCR for Fragile X and Sex Chromosome Aneuploidies

### Materials and Methods

Sodium bisulfite treatment of genomic DNA and/or dried bloodspots was performed as described in Example 1.

### Methylation-Specific Primer Design for FMR1 Promoter:

Two sets of primers were designed, one set specific for the methylated version of the primer sequence, and one specific for the unmethylated version. The primer pair for the methylated version was: FMR1MethFHex 5′-CGC GTT TGT TTT TCG ATT CGG TAT TTC GGT C-3′ (SEQ ID NO: 18) and FMR1MethR 5′-CTC CAC CGA AAA TAA AAC CGA AAC GAA ACT AAA CG-3′ (SEQ ID NO: 19). The primer pair for the methylated version was: FMR1UnmethFHex 5′-TGG TTT GTG TGT TTG TTT TTT GAT TTG GTA TTT TGG TT-3′ (SEQ ID NO: 20) and FMR1UnmethR 5′-ACC CAC ACT CAC CAT CAA CCC ACC A-3′ (SEQ ID NO: 21). A primer pair was also designed for the SRY gene of the Y chromosome: SRYNaHSO3F 5′-ATT AGT AAG TAG TTG GGA TAT TAG TGG AAA ATG TTT ATT G-3′ (SEQ ID NO: 22) and SRYNaHSO3R 5′-TAT AAC TTT CRT ACA ATC ATC CCT ATA CAA CCT ATT ATC C-3′ (SEQ ID NO: 23).

PCR was performed as described in the Examples above, with the exception that Taqman probes were not added. After PCR a portion of the PCR products are diluted in formamide and loaded on an ABI3100 to separate the PCR products by capillary electrophoresis. methylated to unmethylated FMR1 DNA. Quantitation of amount methylated and unmethylated FMR1 DNA allows the distinction between normal females and fragile X females.

### Analysis

The results from the present Example are not shown, but are presented in the copending U.S. provisional patent application Ser. No. 60/724,633, which is incorporated herein by reference. The peak specific for methylated FMR1 DNA is 223 bp. The peak specific for unmethylated FMR1 DNA is 271 bp. The SRY-specific product is 249 bp. The areas underneath the peak from the electropherogram are calculated using the Genescan fragment analysis software. This area is proportional to the amount of methylated and unmethylated FMR1 DNA and SRY DNA present in the original sample.

In addition to FMR1, a probe for the SRY gene, located on the Y chromosome, is included. The presence or absence of the Y chromosome verifies the gender of the individual. In addition, the amount of SRY signal is quantified to distinguish between one or multiple copies of the Y chromosome in a sample. This is important in a population screen, such as newborn screening, since the incidence of sex chromosome aneuploidies, abnormal numbers of sex chromosomes, is more frequent than fragile X syndrome.

### Males

In a normal male a 271 bp product (unmethylated FMR1) and a 249 bp product (SRY) would be detected. In a fragile X male a 223 bp product (methylated FMR1) and a 249 bp product (SRY) would be detected. Mosaic fragile X males can also be detected by the presence of signal from both methylated and unmethylated FMR1 DNA along with the SRY signal.

### Females

In a normal female both a 223 bp product and a 271 bp product will be present in a one to one ratio. The presence of the methylated FMR1 product is due to the normal process of random X inactivation, which results in one of the two copies of FMR1 DNA to become methylated. In a fragile X female a skewing of this one-to-one ratio to a three-to-one ratio of methylated to unmethylated FMR1 DNA will be detected. Quantitation of amount methylated and unmethylated FMR1 DNA allows the distinction between normal females and fragile X females.

### Sex Chromosome Abnormalities

For sex chromosome abnormalities a variety of combinations of the sex chromosomes can be detected depending upon the exact combination and numbers of sex chromosomes present in a sample. For example, when methylated and unmethylated FMR1 DNA is detected along with the SRY gene this is a pattern indicating that the individual, who is phenotypically male, has Klinefelter syndrome. Turner syndrome will be indicated in an individual who is phenotypically female when only a single unmethylated FMR1 gene is detected without any signal for SRY. Additional copies of the sex chromosomes will be detected as alterations in the signal intensities from both the X chromosome probe (the FMR1 gene) and the Y chromosome probe (SRY).

We claim:

1. A method comprising:
contacting a nucleic acid containing sample from a subject with an agent that modifies unmethylated cytosine;
amplifying a target nucleic acid sequence; and
quantifying an amount of a methylated version of the target nucleic acid sequence and an amount of an unmethylated version of the target nucleic acid sequence with probes capable of distinguishing between the target nucleic acid sequence containing methylated cytosines and the target nucleic acid sequence not containing methylated cytosines.

2. The method of claim 1, wherein the cytosines are converted to uracil.

3. The method of claim 2, wherein the agent is sodium bisulfate.

4. The method of claim 3, wherein the agent further comprises hydroquinone.

5. The method of claim 1, wherein the target nucleic acid sequence is amplified by polymerase chain reaction with primers specific for the target nucleic acid sequence.

6. The method of claim 5, wherein the polymerase chain reaction comprises real-time polymerase chain reaction.

7. The method of claim 1, wherein the probes comprise:
a first probe specific for the unmethylated target nucleic acid sequence and having a first reporter molecule and a quencher molecule; and
a second probe specific for the methylated target nucleic acid sequence and having a second reporter molecule and a quencher molecule,
wherein the first and second reporter molecules are distinguishable.

8. The method of claim 7, wherein quantifying the amount of the methylated and unmethylated versions of the target nucleic acid sequence comprises correlating an intensity of a signal produced by the first reporter molecule to an amount of the unmethylated target sequence and correlating an intensity of a signal produced by the second reporter molecule to an amount of the methylated target sequence.

9. The method of claim 1, wherein the nucleic acid containing sample is a blood or tissue sample.

10. The method of claim 9, wherein the sample is a blood spot on filter paper.

11. The method of claim 1, further comprising screening for a condition associated with abnormal methylation of a target nucleic acid sequence in a specific gene indicated by the amount of a methylated version of the target nucleic acid.

12. The method of claim 11, wherein the condition is fragile X syndrome.

13. The method of claim 12, wherein the gene is the FMR1 gene, and the nucleic acid sequence is located in the promoter region of the FMR1 gene.

14. The method of claim 13, wherein the probes comprise at lease one probe pair specific for a nucleic acid sequence corresponding to a sequence in the promoter region of the FMR1 gene, and wherein the probe pair comprises a first probe specific for an unmethylated version of the nucleic acid sequence and a second probe specific for the methylated version of the nucleic acid sequence.

15. The method of claim 1, wherein the subject is a male or female subject.

16. The method of claim 14, wherein the quantifying the amount of the methylated and unmethylated versions of the target nucleic acid present in the sample allows the detection of females with fragile X syndrome and males mosaic for fragile X syndrome.

17. The method of claim 11, wherein the specific gene is selected from at least one of the following: FMR1, SNRPN, H19, and KCNQ10T1.

18. The method of claim 11, wherein the condition is selected from at least one of the following: Fragile X syndrome, Prader-Willi syndrome, Angelman syndrome, autism, Silver Russell syndrome, Beckwith-Wiedemann syndrome, and disorders resulting from sex chromosome aneuploidies.

19. The method of claim 11 comprising screening for more than one condition associated with abnormal nucleic acid methylation in a single assay comprising testing for abnormal methylation in at least two loci on the X chromosome, selected from the following loci: FMR1, SNRPN, H19, and KCNQ10T1.

20. The method of claim 1, wherein the method further comprises quantifying the number of X chromosomes in a subject with probes specific for a nucleic acid sequence located on the X chromosome.

21. The method of claim 1, wherein the method further comprises quantifying the number of Y chromosomes in a subject with probes specific for a nucleic acid sequence located on the Y chromosome.

22. The method of claim 11, wherein samples from more than one subject can be screened in a single assay.

23. The method of claim 22, wherein samples from up to about 100 subjects can be screened in a single assay.

24. A method of screening members of a population for conditions associated with sex chromosome abnormalities comprising:
obtaining samples from one or more subjects regardless of whether said subjects present any symptoms of conditions associated with sex chromosome abnormalities;
contacting the samples with an agent that modifies unmethylated cytosine,
quantifying an amount of a methylated version of at least one target nucleic acid sequence and an amount of an unmethylated version of the at least one target nucleic acid sequence with probes capable of distinguishing between the target nucleic acid sequence containing methylated cytosines and the target nucleic acid sequence not containing methylated cytosines; and
quantifying the number of X and Y chromosomes present in the sample.

25. The method of claim 24, wherein the sex chromosome abnormalities comprise Fragile X syndrome, Prader-Willi syndrome, Angelman syndrome, autism, Silver Russell syndrome, Beckwith-Wiedemann syndrome, and disorders resulting from sex chromosome aneuploidies.

26. The method of claim 25, wherein the disorders resulting from sex chromosome aneuploidies comprise Klinefelter syndrome and its variants, Turner syndrome and its variants, XXX syndrome and its variants, and XYY syndrome and its variants.

27. A kit for screening subjects for fragile X syndrome and sex chromosome aneuploidies comprising:
a primer pair specific for a first nucleic acid sequence in the promoter region of the FMR1 gene; and
a probe pair specific for a second nucleic acid sequence in the promoter region of the FMR1 gene,
wherein the first and second nucleic acid sequences are different, and wherein the probe comprises a first probe specific for an unmethylated version of the second nucleic acid sequence and a second probe specific for a methylated version of the second nucleic acid sequence.

28. The kit of claim 27 further comprising:
a primer pair specific for a first nucleic acid sequence of the SRY gene; and
a probe pair specific for a second nucleic acid sequence of the SRY gene, wherein the first and second nucleic acid sequences are different.

29. The kit of claim 27, further comprising an agent capable of modifying unmethylated cytosines.

30. The kit of claim 29, wherein the agent comprises sodium bisulfite and hydroquinone.

31. The kit of claim 27, wherein the probes are Taqman probes.

32. The kit of claim 27, wherein the first probe comprises SEQ ID NO: 4 and the second probe comprises SEQ ID NO: 3.

33. The kit of claim 27, wherein the primer pair comprises a first primer comprising SEQ ID NO: 1 and a second primer comprising SEQ ID NO: 2.

34. A kit for screening subjects for at least one condition associated with abnormal DNA methylation comprising:
a primer pair specific for a first nucleic acid sequence in the promoter region of a gene associated with the condition; and
a probe pair specific for a second nucleic acid sequence in the promoter region of the gene,
wherein the first and second nucleic acid sequences are different, and wherein the probe pair comprises a first probe specific for an unmethylated version of the second nucleic acid sequence and a second probe specific for a methylated version of the second nucleic acid sequence.

35. The kit of claim 34, wherein the at least one condition is selected from Prader-Willi syndrome, Angelman syndrome, and autism, and wherein the gene is the SNRPN gene.

36. The kit of claim 35, wherein the primer pair comprises a first primer comprising SEQ ID NO: 5 and a second primer comprising SEQ ID NO: 6.

37. The kit of claim 35, wherein the first probe comprises SEQ ID NO: 8 and the second probe comprises SEQ ID NO: 7.

38. The kit of claim 34, wherein the at least one condition is selected from Silver Russell syndrome and Beckwith-Wiedemann syndrome, and wherein the gene is the KCNQ10T1 gene.

39. The kit of claim 38, wherein the primer pair comprises a first primer comprising SEQ ID NO: 9 and a second primer comprising SEQ ID NO: 10.

40. The kit of claim 38, wherein the first probe comprises SEQ ID NO: 12 and the second probe comprises SEQ ID NO: 11.

41. The kit of claim 34, wherein the at least one condition is selected from Silver Russell syndrome and Beckwith-Wiedemann syndrome, and wherein the gene is the H19 gene.

42. The kit of claim 41, wherein the primer pair comprises a first primer comprising SEQ ID NO: 13 and a second primer comprising SEQ ID NO: 14.

43. The kit of claim 41, wherein the first probe comprises SEQ ID NO: 16 and the second probe comprises SEQ ID NO: 15.


SEQUENCE LISTING (23 sequences)

SEQ ID NO: 1 - DNA, 29 bp, Artificial Sequence
  primer
gygtttttaggttatttgaagagagaggg

SEQ ID NO: 2 - DNA, 30 bp, Artificial Sequence
  primer
cracccrctacraatataaacactaaaacc

SEQ ID NO: 3 - DNA, 24 bp, Artificial Sequence
  probe
cggggtcgaggggttgagttcgcg

SEQ ID NO: 4 - DNA, 26 bp, Artificial Sequence
  probe
tggggttgaggggttgagtttgtggg

SEQ ID NO: 5 - DNA, 20 bp, Artificial Sequence
  primer
ggagggagttgggatttttg

SEQ ID NO: 6 - DNA, 20 bp, Artificial Sequence
  primer
acaaacttcrcacacatccc

SEQ ID NO: 7 - DNA, 32 bp, Artificial Sequence
  probe
tgcggtaaataagtacgtttgcgcggtcgtag

SEQ ID NO: 8 - DNA, 45 bp, Artificial Sequence
  probe
tgtggtaaataagtatgtttgtgtggttgtagaggtaggttggtg

SEQ ID NO: 9 - DNA, 21 bp, Artificial Sequence
  primer
gtttaattagtaggtgggggg

SEQ ID NO: 10 - DNA, 27 bp, Artificial Sequence
  primer
cctaacaaaatcttactaaaaaactcc

SEQ ID NO: 11 - DNA, 20 bp, Artificial Sequence
  probe
cggcgggggtagtcggagcg

SEQ ID NO: 12 - DNA, 29 bp, Artificial Sequence
  probe
tggtgggggtagttggagtgttgttgtag

SEQ ID NO: 13 - DNA, 26 bp, Artificial Sequence
  primer
gtatagtatatgggtatttttggagg

SEQ ID NO: 14 - DNA, 28 bp, Artificial Sequence
  primer
cccaattaaaacraactcraactataat

SEQ ID NO: 15 - DNA, 24 bp, Artificial Sequence
  probe
aagtggtcgcgcggcggtagtgta

SEQ ID NO: 16 - DNA, 29 bp, Artificial Sequence
  probe
tggaagtggttgtgtggtggtagtgtagg

SEQ ID NO: 17 - DNA, 150 bp, Homo sapiens
gcagcgcgcatgcgcgcgctcccaggccacttgaagagagagggcggggccgaggggctgagcccgcggggggagggaacagcgttgatcacgtgacgtggtttcagtgtttacacccgcagcgggccgggggttcggcctcaggcgctc

SEQ ID NO: 18 - DNA, 31 bp, Artificial sequence
  primer
cgcgtttgtttttcgattcggtatttcggtc

SEQ ID NO: 19 - DNA, 35 bp, Artificial sequence
  primer
ctccaccgaaaataaaaccgaaacgaaactaaacg

SEQ ID NO: 20 - DNA, 38 bp, Artificial sequence
  primer
tggtttgtgtgtttgttttttgatttggtattttggtt

SEQ ID NO: 21 - DNA, 25 bp, Artificial sequence
  primer
acccacactcaccatcaacccacca

SEQ ID NO: 22 - DNA, 40 bp, Artificial sequence
  primer
attagtaagtagttgggatattagtggaaaatgtttattg

SEQ ID NO: 23 - DNA, 40 bp, Artificial sequence
  primer
tataactttcrtacaatcatccctatacaacctattatcc